(19) United States
(12) Patent Application Publication
(10) Pub. No.: US 2008/0269195 A1
(43) Pub. Date: Oct. 30, 2008

(54) Compounds Having Affinity For the Dopamine D3 Receptor and Uses Thereof in Medicine

(76) Inventors: Giorgio Bonanomi, Verona (IT); Francesca Cardullo, Verona (IT); Federica Damiani, Verona (IT); Gabriella Gentile, Verona (IT); Dieter Hamprecht, Verona (IT); Fabrizio Micheli, Verona (IT); Luca Tarsi, Verona (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/571,051

(22) PCT Filed: Jun. 29, 2005
(86) PCT No.: PCT/EP05/07069

§ 371 Date: Dec. 17, 2007

(30) Foreign Application Priority Data
Jul. 1, 2004 (GB) 0414795.5

(51) Int. Cl. A61K 31/55; C07D 498/04; A61P 25/30

(52) U.S. Cl. 514/215; 540/578

(57) ABSTRACT

Compounds of formula (I) or a salt thereof are disclosed:

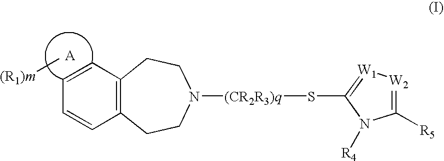

wherein, A, m R₁, R₂, R₃, q, W₁, W₂, R₄ and R₅ are as defined in the description. Processes for preparation and uses of the compounds in medicine, for example for the treatment of schizophrenia or drug dependency, are also disclosed.

The present invention relates to novel compounds, processes for their preparation, intermediates used in these processes, pharmaceutical compositions containing them and their use in therapy, as modulators of dopamine D₃ receptors, in particular as antipsychotic agents or as agents to treat various aspects of drug dependency.

WO 2002/40471 (SmithKline Beecham) discloses certain benzodiazepine compounds having activity at the dopamine D₃ receptor.

A new class of compounds which have affinity for dopamine receptors, in particular the dopamine D₃ receptor, has been found. These compounds have potential in the treatment of conditions wherein modulation, especially antagonism/inhibition, of the D₃ receptor is beneficial, e.g. as antipsychotic agents or to treat drug dependency.

The present invention provides a compound of formula (I) or a salt thereof:

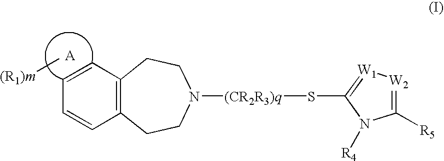

wherein
A is a 5 or 6 membered heteroaromatic ring or a 5 or 6 membered heterocyclic ring;
m is 0, 1, 2 or 3;
R₁ is independently halogen, oxo, hydroxy, cyano, nitro, C₁₋₄alkyl, haloC₁₋₄alkyl, C₃₋₆cycloalkyl, C₁₋₄alkoxy, haloC₁₋₄alkoxy, C₁₋₄alkoxyC₁₋₄alkoxy, C₁₋₄alkylenedioxy, C₁₋₄alkylthio, C₁₋₄alkoxyC₁₋₄alkyl, C₃₋₆cycloalkylC₁₋₄alkoxy, C₃₋₆cycloalkylC₁₋₄alkyl, C₁₋₄alkanoyl, C₁₋₄alkoxycarbonyl, C₁₋₄alkoxycarbonylC₁₋₄alkyl, C₁₋₄alkylsulfonyl, C₁₋₄alkylsulfonyloxy, haloC₁₋₄alkylsulfonyl, haloC₁₋₄alkylsulfonyloxy, C₁₋₄alkylsulfonylC₁₋₄alkyl, C₁₋₄alkylsulfonamido, C₁₋₄alkylsulfonamidoC₁₋₄alkyl, heterocyclyl, aryl, arylC₁₋₄alkoxy, aryloxy, arylthio, arylmethyl, aroyl, aryloxymethyl, arylsulfonyl, aryl-NR′— (wherein R′ is hydrogen or C₁₋₄alkyl), arylsulfonyloxy, arylsulfonylC₁₋₄alkyl, arylsulfonamido, arylcarboxamido, arylsulfonamidoC₁₋₄alkyl, arylcarboxamidoC₁₋₄alkyl, aroylC₁₋₄alkyl, arylClalkanoyl, a group NR₆R₇, R₆CON(R₇)(CH₂)ᵣ, R₆R₇NCO(CH₂)ᵣ or R₆R₇NSO₂(CH₂)ᵣ (in which r is 0, 1, 2, 3 or 4, and each of R₆ and R₇ is independently hydrogen or C₁₋₄alkyl, or in the groups NR₆R₇, R₆CON(R₇)(CH₂)ᵣ, R₆R₇NCO(CH₂)ᵣ and R₆R₇NSO₂(CH₂)ᵣ, R₆CONR₇ or NR₆R₇ together form a 4-, 5-, 6- or 7-membered azacyclic group optionally containing one additional O, N or S atom in the azacycle and having 3-8 carbon atoms (including the carbon atoms contained in any optional substituent(s) of the azacycle)); wherein in any group containing an aryl moiety, the aryl moiety is optionally substituted by one, two or three substituents selected from the group consisting of halogen, hydroxy, cyano, nitro, amino, C₁₋₄alkyl, haloC₁₋₄alkyl, C₁₋₄alkoxy, haloC₁₋₄alkoxy, C₁₋₄alkylenedioxy, C₁₋₄alkanoyl, C₁₋₄alkylsulfonyl, haloC₁₋₄alkylsulfonyl, C₁₋₄alkylamino, C₁₋₄dialkylamino, R₈R₉NCO (in which R₈ and R₉ are independently hydrogen or C₁₋₄alkyl, or R₈R₉N together form a 4-, 5-, 6- or 7-membered azacyclic group optionally containing one additional O, N or S atom in the azacycle and having 3-8 carbon atoms (including the carbon atoms contained in any optional substituent(s) of the azacycle));
R₂ and R₃ are independently hydrogen or methyl;
q is 2, 3 or 4;
W₁ and W₂ are independently N, CH or —C(C₁₋₄alkyl)-;
R₄ is hydrogen or C₁₋₄alkyl;
R₅ is a group of the formula (a) or (b):

-z (a)

—(CR₁₀R₁₁)ₜZ (b)
wherein
   Z is C₁₋₄alkyl, haloC₁₋₄alkyl, C₃₋₆cycloalkyl, phenyl, heterocyclyl, a 5- or 6-membered heteroaromatic group or a 8- to 11-membered bicyclic group, any of which is optionally substituted by 1, 2, 3 or 4 substituents selected from the group consisting of: halogen, hydroxy, oxo, cyano, nitro, C₁₋₄alkyl, C₁₋₄alkoxy, haloC₁₋₄alkyl, haloC₁₋₄alkoxy, C₁₋₄alkylenedioxy, C₁₋₄alkanoyl, C₁₋₄alkylsulfonyl, C₁₋₄alkylsulfonyloxy, haloC₁₋₄alkylsulfonyl, haloC₁₋₄alkylsulfonyloxy, C₁₋₄alkylsulfinyl, C₁₋₄alkylthio, R₁₂SO₂NR₁₃—, R₁₂R₁₃NSO₂—, R₁₂R₁₃N—, R₁₂R₁₃NCO—, R₁₂CONR₁₃— and a 5- or 6-membered heteroaromatic group which is optionally substituted by one or two groups selected from C₁₋₂alkyl, haloC₁₋₂alkyl and R₁₂R₁₃N—; and wherein substituents positioned ortho to one another may be linked to form a 5- or 6-membered ring;
   R₁₀ and R₁₁ are independently hydrogen or C₁₋₄alkyl and t is 1, 2, 3 or 4, or —(CR₁₀R₁₁)ₜ— forms a C₃₋₆cycloalkylene linker; and
   R₁₂ and R₁₃ are independently hydrogen or C₁₋₄alkyl, or R₁₂ and R₁₃ together form C₃₋₆alkylene.

In formula (I), “—S—” means thio (sulfur).

The term “5- or 6-membered heteroaromatic ring” refers to a monocyclic 5- or 6-membered aromatic heterocyclic ring containing 1, 2, 3 or 4 heteroatoms, for example from 1 to 3 heteroatoms, selected from O, N and S. When the group contains 2-4 heteroatoms, one may be selected from O, N and S and the remaining heteroatoms may be N. Examples of 5 and 6-membered heteroaromatic groups include pyrrolyl, pyrrolinyl, pyrazolinyl, imidazolyl, pyrazolyl, oxazolyl, isoxazolyl, oxadiazolyl, isothiazolyl, thiazolyl, furyl, thienyl, thiadiazolyl, pyridyl, triazolyl, thiazinyl, triazinyl, pyridazinyl, pyrimidinyl and pyrazinyl.

The term “5- or 6-membered heterocyclic ring” refers to a 5 or 6-membered monocyclic ring which is partially or fully saturated, and wherein 1, 2, 3, 4 or 5 of the carbon atoms are replaced by a heteroatom independently selected from O, S and N. Examples of “heterocyclyl” which are fully saturated 5 or 6-membered monocyclic rings include pyrrolidinyl, imidazolidinyl, pyrazolidinyl, tetrahydrofuranyl, dioxolanyl, piperidinyl, piperazinyl, morpholinyl, thiomorpholinyl, tetrahydrothienyl, dioxanyl, tetrahydro-2H-pyranyl and dithianyl. Examples of “heterocyclyl” groups which are partially saturated 5 or 6-membered monocyclic rings include oxazolinyl, isoaxazolinyl, imidazolinyl, pyrazolinyl, 1,2,3,6-tetrahydropyridyl and 3,6-dihydro-2H-pyranyl.

The term “C₁₋₄alkyl” refers to an alkyl group having from one to four carbon atoms, in all isomeric forms, such as methyl, ethyl, propyl, isopropyl, butyl, isobutyl, sec-butyl and tert-butyl. “C₁₋₆alkyl” includes, in addition to the above, groups such as pentyl and hexyl.

The term “alkylene” refers to a straight or branched chain divalent hydrocarbon radical. Examples of C₁₋₃alkylene groups include methylene, ethylene and n-propylene. Examples of “C₁₋₄alkylene” include, in addition to the above, n-butylene.

The term “C₁₋₄alkoxy” refers to a straight chain or branched chain alkoxy (or “alkyloxy”) group having from one to four carbon atoms, such as methoxy, ethoxy, propoxy, isopropoxy, butoxy, isobutoxy, sec-butoxy and tert-butoxy.

The term “halogen” and its abbreviation “halo” refer to fluorine (F), chlorine (Cl), bromine (Br) or iodine (I). Where the term “halo” is used before another group, it indicates that the group is substituted by one, two or three halogen atoms. For example, “haloC₁₋₄alkyl” refers to groups such as trifluoromethyl, bromoethyl, trifluoropropyl, and other groups derived from C₁₋₄alkyl groups as defined above; and the term “haloC₁₋₄alkoxy” refers to groups such as trifluoromethoxy, bromoethoxy, trifluoropropoxy, and other groups derived from C₁₋₄alkoxy groups as defined above.

The term “C₁₋₄alkoxyC₁₋₄alkyl” refers to a C₁₋₄alkoxy group attached through a C₁₋₄alkylene group, for example methoxymethyl, ethoxymethyl, propoxyethyl, isopropoxyethyl and others derived from the C₁₋₄alkoxy and C₁₋₄alkyl groups as defined above.

The term “C₁₋₄alkylthio” refers to a C₁₋₄alkyl group attached through a sulfur atom (—S—). Examples of C₁₋₄alkylthio include methylthio, ethylthio, propylthio and butylthio.

The term “C₃₋₆cycloalkyl” refers to a cycloalkyl group having from three to six carbon atoms, such as cyclopropyl, cyclobutyl, cyclopentyl and cyclohexyl. The term “C₃₋₆cycloalkylene” refers to a divalent cycloalkyl group, such as cyclopropylene, cyclobutylene, cyclopentylene and cyclohexylene.

The term “C₃₋₆cycloalkylC₁₋₄alkyl” refers to a cycloalkyl group attached through a C₁₋₄alkylene group, such as cyclopropylmethyl, cyclobutylethyl, and others derived from C₃₋₆cycloalkyl groups and C₁₋₄alkyl groups as defined above.

The term “C₃₋₆cycloalkylC₁₋₄alkoxy” refers to a cycloalkyl group attached through a C₁₋₄alkoxy group, such as cyclopropylmethyleneoxy, cyclobutylethyleneoxy, and others derived from C₃₋₆cycloalkyl groups and C₁₋₄alkoxy groups as defined above.

The term “aryl” refers to phenyl or a 5- or 6-membered heteroaromatic group. Examples of 5- or 6-membered heteroaromatic groups include furanyl, thienyl, pyrrolyl, oxazolyl, thiazolyl, imidazolyl, oxadiazolyl, thiadiazolyl, pyridinyl, triazolyl, triazinyl, pyridazyl, pyrimidinyl, isothiazolyl, isoxazolyl, pyrazinyl, pyrazolyl and pyrimidinyl

The term “arylC₁₋₄alkyl” refers to an aryl group attached through a C₁₋₄alkylene group. The C₁₋₆alkylene group may be in any suitable isomeric form. Examples of arylC₁₋₄alkyl include benzyl, phenethyl (including phenyl-CH₂CH₂— and phenyl-C(CH₃)—) and others derived from the aryl groups and C₁₋₄alkyl groups as defined above.

The terms “arylC₁₋₄alkoxy” refers to an aryl group attached through a C₁₋₄alkoxy group. Examples of arylC₁₋₄alkoxy include benzyloxy (phenyl-CH₂O—) and phenylethoxy.

The term “sulfonyl” refers to the group —SO₂—. Thus, the term “C₁₋₄alkylsulfonyl” includes methylsulfonyl, ethylsulfonyl, and others derived from the C₁₋₄alkyl groups defined above.

The term “haloC₁₋₄alkylsulfonyl” refers to groups such as trifluoromethanesulfonyl and pentafluoroethylsulfonyl. The term “arylsulfonyl” includes phenylsulfonyl, pyridinylsulfonyl, and others derived from aryls as defined above.

The term “arylcarboxamido” refers to groups such as phenylcarboxamido and pyridinylcarboxamido, and others derived from the aryl groups as defined above.

The term “C₁₋₄alkylenedioxy” refers to groups such as methylenedioxy, ethylenedioxy and others derived from C₁₋₄alkyl as defined above.

The term “8- to 11-membered bicyclic group” refers to a bicyclic ring system containing a total of 8, 9, 10 or 11 carbon atoms, wherein 1, 2, 3 or 4 or 5 of the carbon atoms are optionally replaced by a heteroatom independently selected from O, S and N. The term includes bicyclic systems wherein both rings are aromatic, as well as bicyclic ring systems wherein one of the rings is partially or fully saturated. Examples of 8- to 11-membered bicyclic groups wherein both rings are aromatic include indenyl, naphthyl and azulenyl. Examples of 8- to 11-membered bicyclic groups having 1, 2, 3, 4 or 5 heteroatoms, in which both rings are aromatic, include: 6H-thieno[2,3-b]pyrrolyl, imidazo[2,1-b][1,3]thiazolyl, imidazo[5,1-b][1,3]thiazolyl, [1,3]thiazolo[3,2-b][1,2,4]triazolyl, indolyl, isoindolyl, indazolyl, benzimidazolyl e.g. benzimidazol-2-yl, benzoxazolyl e.g. benzoxazol-2-yl, benzisoxazolyl, benzothiazolyl, benzisothiazolyl, benzothienyl, benzofuranyl, naphthridinyl, quinolyl, quinoxalinyl, quinazolinyl, cinnolinyl and isoquinolyl. Examples of 8- to 11-membered bicyclic groups having 1, 2, 3, 4 or 5 heteroatoms, in which one of the rings is partially or fully saturated includes dihydrobenzofuranyl, indanyl, tetrahydronaphthyl, indolinyl, isoindolinyl, tetrahydroisoquinolinyl, tetrahydroquinolyl, benzoxazinyl and benzoazepinyl.

The term “8- to 11-membered bicyclic heterocyclic group” refers to a bicyclic ring system containing a total of 8, 9, 10 or 11 carbon atoms, wherein 1, 2, 3, 4 or 5 of the carbon atoms are replaced by a heteroatom independently selected from O, S and N. Examples of 8- to 11-membered bicyclic heterocyclic groups in which both rings are aromatic include: 6H-thieno[2,3-b]pyrrolyl, imidazo[2,1-b][1,3]thiazolyl, imidazo[5,1-b][1,3]thiazolyl, [1,3]thiazolo[3,2-b][1,2,4]triazolyl, indolyl, isoindolyl, indazolyl, benzimidazolyl e.g. benzimidazol-2-yl, benzoxazolyl e.g. benzoxazol-2-yl, benzisoxazolyl, benzothiazolyl, benzisothiazolyl, benzothienyl, benzofuranyl, naphthridinyl, quinolyl, quinoxalinyl, quinazolinyl, cinnolinyl and isoquinolyl. Examples of 8- to 11-membered bicyclic heterocyclic groups, in which one of the rings is partially or fully saturated includes dihydrobenzofuranyl, indanyl, tetrahydronaphthyl, indolinyl, isoindolinyl, tetrahydroisoquinolinyl, tetrahydroquinolyl, benzoxazinyl and benzoazepinyl.

The term “heterocyclyl” refers to a 5 or 6-membered monocyclic or 8 to 11-membered bicyclic group which is partially or fully saturated, wherein 1, 2, 3, 4 or 5 of the carbon atoms are replaced by a heteroatom independently selected from O, S and N. Examples of “heterocyclyl” which are fully saturated 5 or 6-membered monocyclic rings include pyrrolidinyl, imidazolidinyl, pyrazolidinyl, tetrahydrofuranyl, dioxolanyl, piperidinyl, piperazinyl, morpholinyl, thiomorpholinyl, tetrahydrothienyl, dioxanyl, tetrahydro-2H-pyranyl and dithianyl. Examples of “heterocyclyl” groups which are partially saturated 5 or 6-membered monocyclic rings include oxazolinyl, isoaxazolinyl, imidazolinyl, pyrazolinyl, 1,2,3,6-tetrahydropyridyl and 3,6-dihydro-2H-pyranyl. Examples of “heterocyclyl” groups which are fully saturated 8 to 11-membered bicyclic rings include decahydroquinolinyl, octahydro-2H-1,4-benzoxazinyl and octahydro-1H-cyclopenta[b]pyridinyl. Examples of “heterocyclyl” groups which are partially saturated 8 to 11-membered bicyclic rings include 2,3-dihydro-1H-indolyl, 1,2,3,4-tetrahydroquinolinyl, 1,2,3,4-tetrahydroisoquinolinyl and 2,3,4,5-tetrahydro-1H-3-benzazepinyl.

Any of these groups may be attached to the rest of the molecule at any suitable position.

As used herein, the term “salt” refers to any salt of a compound according to the present invention prepared from an inorganic or organic acid or base, quaternary ammonium salts and internally formed salts. Physiologically acceptable salts are particularly suitable for medical applications because of their greater aqueous solubility relative to the parent compounds. Such salts must clearly have a physiologically acceptable anion or cation. Suitably physiologically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-physiologically acceptable anion or cation are within the scope of the invention as useful intermediates for the preparation of physiologically acceptable salts and/or for use in non-therapeutic, for example, in vitro, situations.

In one embodiment, m is 0 or 1.

When R₁ contains an aryl moiety, ie R₁ is aryl, arylC₁₋₄alkoxy, aryloxy, arylthio, arylmethyl, aroyl, aryloxymethyl, arylsulfonyl, aryl-NR′—, arylsulfonyloxy, arylsulfonylC₁₋₄alkyl, arylsulfonamido, arylcarboxamido, arylsulfonamidoC₁₋₄alkyl, arylcarboxamidoC₁₋₄alkyl, aroylC₁₋₄alkyl or arylC₁₋₄alkanoyl, the aryl moiety is optionally substituted by one or two substituents selected from: halogen, cyano, C₁₋₂alkyl (e.g. methyl), fluoroC₁₋₂alkyl (eg trifluoromethyl), C₁₋₂alkoxy (e.g. methoxy), C₁₋₂alkylenedioxy (e.g. methylenedioxy), C₁₋₃alkanoyl (e.g. acetyl), C₂alkanoylamino (e.g. acetylamino), fluoroC₁alkylsulfonyl (e.g. trifluoromethylsulfonyl) and methylsulfonyl. For example, the aryl moiety is optionally substituted by methyl.

When R₁ is a group NR₆R₇, R₆CON(R₇)(CH₂)ᵣ, R₆R₇NCO(CH₂)ᵣ or R₆R₇NSO₂(CH₂)ᵣ and R₆CONR₇ or R₆R₇N together form a 4-, 5-, 6- or 7-membered azacyclic group, then this is characterised by: (i) containing one additional O, N or S atom in the azacycle, for example the azacyclic group being 1,4-morpholin-4-yl and/or (ii) having 1 or 2 optional C₁₋₂alkyl substituents whose carbon atoms are included in the azacyclic group's 3-8 carbon atoms. One, two or more F atoms can optionally be included as substituents of the carbon atoms of the heterocycle. The term “azacyclic group” should be interpreted to cover only stable azacycles such as 1,4-morpholine and piperazine and not for example 1,3-morpholine. Saturated azacycles, in particular piperidinyl, pyrrolidinyl, 1,4-morpholinyl, and including the corresponding α-oxo-azacycles R₆CONR₇, may be given as examples.

In one embodiment, R₁ is halogen, oxo, cyano, C₁₋₄alkyl (such as methyl, ethyl, propyl, isopropyl, butyl, tert-butyl), haloC₁₋₄alkyl (such as —CF₃, —CH₂CF₃, —CF₂CH₃ or pentafluoroethyl), acetyl, trifluoromethoxy, C₃₋₆cycloalkylC₁₋₄alkyl (such as cyclopropylmethyl), C₃₋₆cycloalkyl (such as cyclopropyl), C₁₋₄alkylsulfonyl, C₁₋₄alkylsulfonyloxy, R₆R₇NSO₂ (where each of R₆ and R₇ is independently hydrogen or C₁₋₄alkyl or R₆R₇N together form a 4-, 5-, 6- or 7-membered azacyclic group optionally containing one additional O, N or S atom in the azacycle and having 3-8 carbon atoms), a heterocyclyl, or a 5- or 6-membered heteroaromatic group which is optionally substituted by one or two substituents selected from: halogen, cyano, C₁₋₂alkyl (e.g. methyl), haloC₁₋₂alkyl (e.g. trifluoromethyl), C₁₋₂alkoxy (e.g. methoxy), C₁₋₂alkylenedioxy (e.g. methylenedioxy), C₁₋₃alkanoyl (e.g. acetyl), C₂alkanoylamino (e.g. acetylamino), haloC₁alkylsulfonyl (e.g. trifluoromethylsulfonyl) and methylsulfonyl.

For example, R₁ is selected from: halogen, C₁₋₄alkylsulfonyl (e.g. methylsulfonyl or ethylsulfonyl), haloC₁₋₄alkylsulfonyl (e.g. trifluoromethylsulfonyl), C₁₋₄alkylsulfonyloxy (e.g. methylsulfonyloxy), haloC₁₋₄alkylsulfonyloxy (e.g. trifluoromethylsulfonyloxy), R₆R₇NSO₂ (where each of R₆ and R₇ is independently hydrogen or C₁₋₄alkyl, e.g. N,N-dimethylaminosulfonyl, or where R₆R₇N together form a 4-, 5-, 6- or 7-membered azacyclic group optionally containing one additional O, N or S atom in the azacycle and having 3-8 carbon atoms, e.g. a piperidin-1-ylsulfonyl, pyrrolidin-1-ylsulfonyl or 1,4-morpholin-4-ylsulfonyl), a 5- or 6-membered heteroaromatic or a heterocyclyl, each of which is optionally substituted by one or two substituents selected from: halogen, cyano, C₁₋₂alkyl (e.g. methyl or trifluoromethyl), C₁₋₂alkoxy (e.g. methoxy), C₁₋₂alkylenedioxy (e.g. methylenedioxy), C₁₋₃alkanoyl (e.g. acetyl), C₂alkanoylamino (e.g. acetylamino), haloC₁alkylsulfonyl (e.g. trifluoromethylsulfonyl) and methylsulfonyl.

Suitably, R₁ is bromo, cyano, hydroxy, chloro, methoxy, tert-butyl, methylsulfonyl, ethylsulfonyl, N,N-dimethylaminosulfonyl, pyrrolidin-1-ylsulfonyl, 1,4-morpholin-4-ylsulfonyl, methylsulfonyloxy, pyrazin-2-yl, 5-methyl-oxazol-2-yl or 5-methyl-isoxazol-3-yl.

In one embodiment, R₁ is C₁₋₄alkyl or haloC₁₋₄alkyl and m is 0, 1 or 2.

In one embodiment, A is a 5- or 6-membered heteroaromatic ring such as for example oxazole or thiazole.

In one embodiment, R₂ and R₃ are hydrogen at each occurrence.

In one embodiment, q is 2 or 3.

In one embodiment, W. and W₂ are both N.

In one embodiment, R₄ is hydrogen or methyl.

When R₅ is a group of formula (a), it may be for example phenyl, a bicyclic heterocyclic group or a 5- or 6-membered heteroaromatic group, each of which is optionally substituted by one or two groups selected from C₁₋₂alkyl and haloC₁₋₂alkyl.

For example, R₅ may be phenyl (such as unsubstituted phenyl or fluorophenyl (e.g. 4-fluorophenyl)), optionally substituted quinolinyl (e.g. 2-, 3-, 4-, 5- or 6-quinolinyl), furyl (e.g. 2-furyl), oxazolyl (e.g. 5-oxazolyl), thiazolyl (e.g. 5-thiazolyl), thienyl (e.g. 2-thienyl), pyridyl (e.g. 4-pyridyl), indolyl, pyrazolopyrimidyl (e.g. pyrazolo[1,5-a]pyrimidyl), cinnolinyl, benzo[b]furanyl, thienopyridine or pyrrolopyridyl.

When R₅ is a group of formula (b), examples include —(CH₂)-Z, —(CHCH₃)-Z and groups such as:

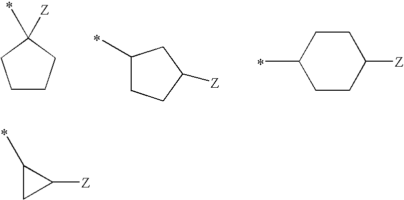

Examples of Z in formula (b) include those given for Z in formula (a).

In one embodiment, the present invention provides a compound of formula (Ia):

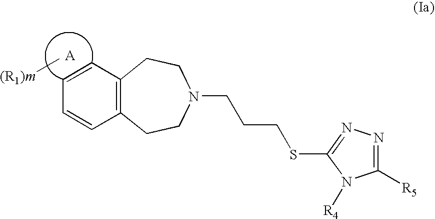

wherein

A is a 5 or 6 membered heteroaromatic ring;

m is 0, 1 or 2;

R₁ is C₁₋₄alkyl or haloC₁₋₄alkyl;

R₄ is hydrogen or methyl;

R₅ is phenyl, a 5 or 6-membered heteroaromatic group or a 8-11 membered bicyclic group, each of which is optionally substituted by one or two groups selected from C₁₋₂alkyl and haloC₁₋₂alkyl.

All features and embodiments of the compounds of formula (I) apply to formula (Ia) mutatis mutandis.

Examples of the compounds of the present invention include:
2-methyl-8-(3-{[4-methyl-5-(2-methyl-5-quinolinyl)-4H-1,2,4-triazol-3-yl]thio}propyl)-7,8,9,10-tetrahydro-6H-[1,3]oxazolo[4,5-g][3]benzazepine
2-ethyl-8-(3-{[4-methyl-5-(2-methyl-5-quinolinyl)-4H-1,2,4-triazol-3-yl]thio}propyl)-7,8,9,10-tetrahydro-6H-[1,3]oxazolo[4,5-g][3]benzazepine
2-ethyl-8-(3-{[4-methyl-5-(4-methyl-1,3-oxazol-5-yl)-4H-1,2,4-triazol-3-yl]thio}propyl)-7,8,9,10-tetrahydro-6H-[1,3]oxazolo[4,5-g][3]benzazepine
8-(3-{[4-methyl-5-(2-methyl-5-quinolinyl)-4H-1,2,4-triazol-3-yl]thio}propyl)-2-(trifluoromethyl)-7,8,9,10-tetrahydro-6H-[1,3]oxazolo[4,5-g][3]benzazepine
8-(3-{[4-methyl-5-(4-methyl-1,3-oxazol-5-yl)-4H-1,2,4-triazol-3-yl]thio}propyl)-2-(trifluoromethyl)-7,8,9,10-tetrahydro-6H-[1,3]oxazolo[4,5-g][3]benzazepine
2-ethyl-8-(3-{[4-methyl-5-(2-methyl-5-quinolinyl)-4H-1,2,4-triazol-3-yl]thio}propyl)-7,8,9,10-tetrahydro-6H-[1,3]thiazolo[5,4-g][3]benzazepine
2-(1,3-dimethyl-1H-pyrazol-5-yl)-8-(3-{[4-methyl-5-(2-methyl-5-quinolinyl)-4H-1,2,4-triazol-3-yl]thio}propyl)-7,8,9,10-tetrahydro-6H-[1,3]oxazolo[4,5-g][3]benzazepine
2-(1,3-dimethyl-1H-pyrazol-5-yl)-8-(3-{[4-methyl-5-(4-methyl-1,3-oxazol-5-yl)-4H-1,2,4-triazol-3-yl]thio}propyl)-7,8,9,10-tetrahydro-6H-[1,3]oxazolo[4,5-g][3]benzazepine
8-(3-{[4-methyl-5-(2-methyl-5-quinolinyl)-4H-1,2,4-triazol-3-yl]thio}propyl)-2-(pentafluoroethyl)-7,8,9,10-tetrahydro-6H-[1,3]oxazolo[4,5-g][3]benzazepine
8-(3-{[4-methyl-5-(4-methyl-1,3-oxazol-5-yl)-4H-1,2,4-triazol-3-yl]thio}propyl)-2-(pentafluoroethyl)-7,8,9,10-tetrahydro-6H-[1,3]oxazolo[4,5-g][3]benzazepine
8-(3-{[4-methyl-5-(2-methyl-5-quinolinyl)-4H-1,2,4-triazol-3-yl]thio}propyl)-2-(trifluoromethyl)-7,8,9,10-tetrahydro-6H-[1,3]oxazolo[5,4-g][3]benzazepine
8-(3-{[4-methyl-5-(4-methyl-1,3-oxazol-5-yl)-4H-1,2,4-triazol-3-yl]thio}propyl)-2-(trifluoromethyl)-7,8,9,10-tetrahydro-6H-[1,3]oxazolo[5,4-g][3]benzazepine
2-(1,1-difluoroethyl)-8-(3-{[4-methyl-5-(4-methyl-1,3-oxazol-5-yl)-4H-1,2,4-triazol-3-yl]thio}propyl)-7,8,9,10-tetrahydro-6H-[1,3]oxazolo[4,5-g][3]benzazepine
2-(1,1-difluoroethyl)-8-(3-{[4-methyl-5-(2-methyl-5-quinolinyl)-4H-1,2,4-triazol-3-yl]thio}propyl)-7,8,9,10-tetrahydro-6H-[1,3]oxazolo[5,4-g][3]benzazepine
2-(1,1-difluoroethyl)-8-(3-{[4-methyl-5-(4-methyl-1,3-oxazol-5-yl)-4H-1,2,4-triazol-3-yl]thio}propyl)-7,8,9,10-tetrahydro-6H-[1,3]oxazolo[5,4-g][3]benzazepine
8-(3-{[4-methyl-5-(5-methyl-2-pyrazinyl)-4H-1,2,4-triazol-3-yl]thio}propyl)-2-(trifluoromethyl)-7,8,9,10-tetrahydro-6H-[1,3]oxazolo[4,5-g][3]benzazepine
8-(3-{[4-methyl-5-(6-methyl-3-pyridinyl)-4H-1,2,4-triazol-3-yl]thio}propyl)-2-(trifluoromethyl)-7,8,9,10-tetrahydro-6H-[1,3]oxazolo[4,5-g][3]benzazepine
8-(3-{[4-methyl-5-(2-methyl-3-pyridinyl)-4H-1,2,4-triazol-3-yl]thio}propyl)-2-(trifluoromethyl)-7,8,9,10-tetrahydro-6H-[1,3]oxazolo[4,5-g][3]benzazepine
8-{3-[(4-methyl-5-phenyl-4H-1,2,4-triazol-3-yl)thio]propyl}-2-(trifluoromethyl)-7,8,9,10-tetrahydro-6H-[1,3]oxazolo[4,5-g][3]benzazepine
8-(3-{[5-(2,4-dimethyl-1,3-thiazol-5-yl)-4-methyl-4H-1,2,4-triazol-3-yl]thio}propyl)-2-(trifluoromethyl)-7,8,9,10-tetrahydro-6H-[1,3]oxazolo[4,5-g][3]benzazepine
2-methyl-8-(3-{[4-methyl-5-(2-methyl-5-quinolinyl)-4H-1,2,4-triazol-3-yl]thio}propyl)-7,8,9,10-tetrahydro-6H-[1,3]thiazolo[5,4-g][3]benzazepine
2-ethyl-8-(3-{[4-methyl-5-(4-methyl-1,3-oxazol-5-yl)-4H-1,2,4-triazol-3-yl]thio}propyl)-7,8,9,10-tetrahydro-6H-[1,3]thiazolo[5,4-g][3]benzazepine
   and salts thereof.

It will be appreciated that for use in medicine the salts of the compounds of the invention should be pharmaceutically (i.e. physiologically) acceptable. Suitable pharmaceutically acceptable salts will be apparent to those skilled in the art and include for example acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Other non-pharmaceutically acceptable salts e.g. oxalates, may be used, for example in the isolation of compounds of the invention and are included within the scope of this invention. Also included within the scope of the invention are solvates, hydrates, complexes and prodrugs of compounds of the invention. Pharmaceutical acceptable salts may also be prepared from other salts, including other pharmaceutically acceptable salts, of the compound of formula (I) using conventional methods.

Certain of the compounds of the invention may form acid addition salts with less than one equivalent of the acid, or one or more equivalents of the acid. The present invention includes within its scope all possible stoichiometric and non-stoichiometric forms.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention. The compounds of formula (I) may readily be isolated in association with solvent molecules by crystallisation or evaporation of an appropriate solvent to give the corresponding solvates.

In addition, prodrugs are also included within the context of this invention. As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of structure (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

Furthermore, some of the crystalline forms of the compounds of structure (I) may exist as polymorphs, which are included in the present invention.

Those skilled in the art will appreciate that in the preparation of the compound of the invention or a solvate thereof it may be necessary and/or desirable to protect one or more sensitive groups in the molecule to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl type protecting groups (e.g. benzyl, trityl, chlorotrityl). Examples of suitable oxygen protecting groups may include for example alky silyl groups, such as trimethylsilyl or tert-butyldimethylsilyl; alkyl ethers such as tetrahydropyranyl or tert-butyl; or esters such as acetate.

The subject invention also includes isotopically-labelled compounds, which are identical to those recited in formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labelled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I and following of this invention can generally be prepared by carrying out the procedures disclosed in the Schemes and/or in the Examples below, by substituting a readily available isotopically labelled reagent for a non-isotopically labelled reagent.

Certain groups/substituents included in the present invention may be present as isomers. The present invention includes within its scope all such isomers, including racemates, enantiomers, tautomers and mixtures thereof. Certain of the substituted heteroaromatic rings included in compounds of formula (I) may exist in one or more tautomeric forms. The present invention includes within its scope all such tautomeric forms, including mixtures.

In one embodiment of the present invention compounds are provided having a molecular weight of 800 or less. In another embodiment compounds are provided having a molecular weight of 600 or less. Generally, and without being limited thereto, such compounds may have higher oral bioavailability, and sometimes higher solubility and/or brain penetrancy. Molecular weight here refers to that of the unsolvated free base compound, excluding any molecular weight contributed by addition salts, solvent (e.g. water) molecules, prodrug molecular parts cleaved off in vivo, etc.

In general, the compounds or salts of the invention should be interpreted as excluding those compounds (if any) which are so chemically unstable, either per se or in water, that they are clearly unsuitable for pharmaceutical use through all administration routes, whether oral, parenteral or otherwise. Such compounds are known to the skilled chemist. Prodrugs or compounds which are stable ex vivo and which are convertable in the mammalian (e.g. human) body to the inventive compounds are however included.

The present invention also provides a process for preparing a compound of formula (I), which process comprises

(a) reacting a compound of formula (II):

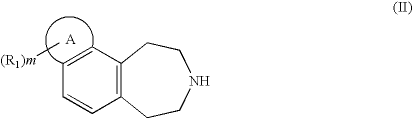

wherein R₁, m and A are as defined for formula (I), with a compound of formula (III):

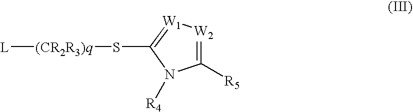

wherein R₂, R₃, q, W₁, W₂, R₄ and R₅ are as defined for formula (I), and L is a leaving group;
or
(b) reacting a compound of formula (IV):

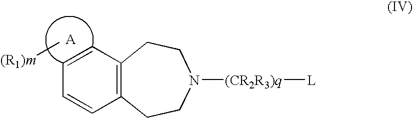

wherein A, R₁, R₂, R₃, m and q are as defined for formula (I) and L is a leaving group, with a compound of formula (V):

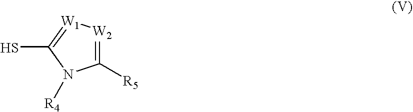

wherein W₁, W₂, R₄ and R₅ are as defined for formula (I);
and optionally thereafter for step (a) or step (b):

removing any protecting group(s); and/or

forming a salt; and/or

converting one compound of formula (I) to a different compound of formula (I).

In step (a), the leaving group, L, in compounds of formula (II) may be for example halogen, such as chlorine. The process of the present invention may be effected using conventional conditions for N-alkylation. For example, when L is a halogen such as chlorine, the reaction may be carried out in the presence of a source of iodide such as sodium iodide using a base such as potassium carbonate in a suitable solvent such as DMF at an appropriate temperature such as around 60° C. Alternatively L may be for example a sulfonyloxy group such as C₁₋₄alkylsulfonyloxy (e.g. methanesulfonyloxy or trifluoromethanesulfonyloxy); or arylsulfonyloxy wherein aryl is optionally substituted phenyl, e.g. para-toluenesulfonyloxy.

In step (b), the leaving group L may be as described for compounds of formula (II) above. It will be appreciated by those skilled in the art that the compound of formula (V) may be replaced by an eventual tautomeric form. For example, when L is a halogen such as chlorine, the reaction may be carried out in the presence of a source of iodide such as sodium iodide using a base such as diisopropylethylamine in a suitable solvent such as DMF at a suitable temperature, for example 80° C.

Compounds of formula (I) may be converted to another compound of formula (I) by suitable methods known to the skilled person, such as:
converting one form of A (e.g. a lactone) to a different form of A (e.g. an N-alkyl lactam); or
replacing one R₁ (e.g. Cl) with a different R₁ (e.g. NR₆R₇).

Compounds of formula (II), (III), (IV) and (V) may be prepared by methods disclosed herein, by methods known in the literature or are commercially available. For example, compounds of formula (VI):

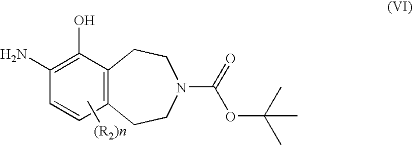

wherein R₂ and n are as defined for formula (I), may be reacted with a carboxylic acid chloride derivative containing an appropriate R₁ group, followed by treatment with trifluoroacetic acid and heating, to form compounds of formula (II) wherein A is 2-substituted oxazole:

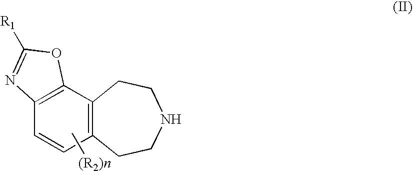

A compound of formula (III) may be prepared by reacting a compound of formula (V) as defined above with a compound of formula (VII):

L-(CR₂R₃)_{q}—X (VII)

wherein R₂, R₃ and q are as defined for formula (I), and L and X are independently leaving groups. Appropriate leaving groups for L and X may be halogen such as Cl or Br, or a sulfonyloxy group such as C₁₋₄alkylsulfonyloxy (e.g. methanesulfonyloxy or trifluoromethanesulfonyloxy); or arylsulfonyloxy wherein aryl is optionally substituted phenyl, e.g. para-toluenesulfonyloxy.

Compounds of formula (IV) may be made for example by reacting a compound of formula (II) with a compound of formula (VIII).

Compounds of formula (I) have been found to exhibit affinity for dopamine receptors, in particular the D₃ receptor, and are expected to be useful in the treatment of disease states which require modulation of such receptors, such as drug dependency or psychotic conditions. Many of the compounds of formula (I) have also been found to have greater affinity for dopamine D₃ than for D₂ receptors.

The therapeutic effect of currently available antipsychotic agents (neuroleptics) is generally believed to be exerted via blockade of D₂ receptors; however this mechanism is also thought to be responsible for undesirable extrapyramidal side effects (eps) associated with many neuroleptic agents. Without wishing to be bound by theory, it has been suggested that blockade of the more recently characterised dopamine D₃ receptor may give rise to beneficial antipsychotic activity without significant eps. (see for example Sokoloff et al, Nature, 1990; 347: 146-151; and Schwartz et al, Clinical Neuropharmacology, Vol 16, No. 4, 295-314, 1993). Preferred compounds of the present invention are therefore those which have higher (e.g. ≧10× or ≧100× higher) affinity for dopamine D₃ than dopamine D₂ receptors (such affinity can be measured using standard methodology for example using cloned dopamine receptors—see herein). Said compounds may advantageously be used as selective modulators of D₃ receptors.

Compounds of formula (I) will be used for treatment of all aspects of drug dependency including prevention of relapse to and relief of withdrawal symptoms from drugs of abuse such as nicotine, alcohol, cocaine, amphetamine, metamphetamine, opiates, benzodiazepines, inhalants and inhibition of tolerance induced by opioids. In addition, compounds of formula (I) and pharmaceutically acceptable salts and solvates thereof will be used to reduce craving and therefore will be useful in the treatment of drug craving. Drug craving can be defined as the incentive motivation to self-administer a psychoactive substance that was previously consumed. Three main factors are involved in the development and maintenance of drug craving: (1) Dysphoric states during drug withdrawal can function as a negative reinforcer leading to craving; (2) Environmental stimuli associated with drug effects can become progressively more powerful (sensitization) in controlling drug seeking or craving, and (3) A cognition (memory) of the ability of drugs to promote pleasurable effects and to alleviate a dysphoric state during withdrawal. Craving may account for the difficulty that individuals have in giving up drugs of abuse and therefore contributes significantly to the maintenance of drug dependence and the probability of relapse or reinstatement of drug seeking and drug taking behaviors.

The compounds of formula (I) are of potential use as antipsychotic agents for example in the treatment of schizophrenia, schizo-affective disorders, psychotic depression, mania, paranoid and delusional disorders. Furthermore, they could have utility as adjunct therapy in Parkinsons Disease, particularly with compounds such as L-DOPA and possibly dopaminergic agonists, to reduce the side effects experienced with these treatments on long term use (e.g. see Schwartz et al., Brain Res. Reviews, 1998, 26, 236-242). From the localisation of D3 receptors, it could also be envisaged that the compounds could also have utility for the treatment of substance abuse where it has been suggested that D3 receptors are involved (e.g. see Levant, 1997, Pharmacol. Rev., 49, 231-252). Examples of such substance abuse include alcohol, cocaine, heroin and nicotine abuse. Other conditions which may be treated by the compounds include dyskinetic disorders such as Parkinson's disease, neuroleptic-induced parkinsonism and tardive dyskinesias; depression; anxiety, cognitive impairment including memory disorders such as Alzheimers disease, eating disorders, sexual dysfunction, sleep disorders, emesis, movement disorders, obsessive-compulsive disorders, amnesia, aggression, autism, vertigo, dementia, circadian rhythm disorders and gastric motility disorders e.g. IBS.

In a further aspect therefore the present invention provides a method of treating a condition for which modulation (especially antagonism/inhibition) of dopamine receptors (especially dopamine D₃ receptors) is beneficial, which comprises administering to a mammal (e.g. human) in need thereof an effective amount of a compound of formula (I) or a pharmaceutically (i.e. physiologically) acceptable salt thereof. Such conditions in particular include psychoses/psychotic conditions such as schizophrenia, and substance abuse and/or drug dependency. For example, the condition to be treated may be craving for abused substance and/or relapse to drug seeking and drug taking behaviour.

The invention also provides the use of a compound of formula (I) or a pharmaceutically acceptable salt thereof in the manufacture of a medicament for the treatment of a condition in a mammal for which modulation (especially antagonism/inhibition) of dopamine receptors (especially dopamine D₃ receptors) is beneficial.

The invention also provides a compound of formula (I) or a pharmaceutically acceptable salt thereof for use in the treatment of a condition in a mammal for which modulation (especially antagonism/inhibition) of dopamine receptors (especially dopamine D₃ receptors) is beneficial.

In one embodiment, D₃ antagonists according to the present invention are used in the treatment of psychoses such as schizophrenia or in the treatment of substance abuse and/or drug dependency.

Thus, a still further aspect the invention provides a method of treating a psychotic condition (e.g. schizophrenia) or substance abuse and/or drug dependency which comprises administering to a mammal (e.g. human) in need thereof an effective amount of a compound of formula (I) as herein defined or a pharmaceutically acceptable salt thereof.

Also provided is the use of a compound of formula (I) or a pharmaceutically acceptable salt thereof in the manufacture of a medicament for the treatment of a psychotic condition (e.g. schizophrenia) or substance abuse and/or drug dependency in a mammal.

Also provided is a compound of formula (I) or a pharmaceutically acceptable salt thereof for use in the treatment of a psychotic condition (e.g. schizophrenia) or substance abuse and/or drug dependency in a mammal.

Also provided is a compound of formula (I) or a pharmaceutically acceptable salt thereof for use as an active therapeutic substance in a mammal, e.g. for use in the treatment of any of the conditions described herein.

“Treatment” and “therapy” include prophylaxis, where this is appropriate for the relevant condition(s).

For use in medicine, the compounds of the present invention are usually administered as a standard pharmaceutical composition. The present invention therefore provides in a further aspect a pharmaceutical composition comprising a compound of formula (I) or a pharmaceutically (i.e. physiologically) acceptable salt thereof and a pharmaceutically (i.e. physiologically) acceptable carrier. The pharmaceutical composition can be for use in the treatment of any of the conditions described herein.

The compounds of formula (I) may be administered by any convenient method, for example by oral, parenteral (e.g. intravenous), buccal, sublingual, nasal, rectal or transdermal administration and the pharmaceutical compositions adapted accordingly.

The compounds of formula (I) and their pharmaceutically acceptable salts which are active when given orally can be formulated as liquids or solids, for example syrups, suspensions or emulsions, tablets, capsules and lozenges.

A liquid formulation will generally consist of a suspension or solution of the compound or pharmaceutically acceptable salt in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluoro-chlorohydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

In one embodiment, the composition is in unit dose form such as a tablet, capsule or ampoule.

Each dosage unit for oral administration contains for example from 1 to 250 mg (and for parenteral administration contains for example from 0.1 to 25 mg) of a compound of the formula (I) or a pharmaceutically acceptable salt thereof calculated as the free base.

The pharmaceutically acceptable compounds of the invention will normally be administered in a daily dosage regimen (for an adult patient) of, for example, an oral dose of between 1 mg and 500 mg, for example between 10 mg and 400 mg, e.g. between 10 and 250 mg or an intravenous, subcutaneous, or intramuscular dose of between 0.1 mg and 100 mg, for example between 0.1 mg and 50 mg, e.g. between 1 and 25 mg of the compound of the formula (I) or a pharmaceutically acceptable salt thereof calculated as the free base, the compound being administered 1 to 4 times per day. Suitably the compounds will be administered for a period of continuous therapy, for example for a week or more.

### Biological Test Methods

Binding Experiments on Cloned Dopamine (e.g. D2, D3 and D4) Receptors

The ability of the compounds to bind selectively to human D2/D3/D4 dopamine receptors can be demonstrated by measuring their binding to cloned receptors. The inhibition constants (Kᵢ) of test compounds for displacement of [¹²⁵I]-Iodosulpride binding to human D2/D3 and [³H]-YM-09151 to D4 dopamine receptors expressed in CHO cells were determined as follows. The cell lines were shown to be free from bacterial, fungal and mycoplasmal contaminants, and stocks of each were stored frozen in liquid nitrogen. Cultures were grown as monolayers or in suspension in standard cell culture media. Cells were recovered by scraping (from monolayers) or by centrifugation (from suspension cultures), and were washed two or three times by suspension in phosphate buffered saline followed by collection by centrifugation. Cell pellets were stored frozen at −80° C. Crude cell membranes were prepared by homogenisation followed by high-speed centrifugation, and characterisation of cloned receptors achieved by radioligand binding.

Preparation of Cho Cell Membranes: Cell Pellets were Gently Thawed at Room temperature, and resuspended in about 20 volumes of ice-cold Extraction buffer; 5 mM EDTA, 50 mM Trizma pre-set crystals (pH7.4@37° C.), 1 mM MgCl₂, 5 mM KCl and 120 mM NaCl. The suspension was homogenised using an Ultra-Turrax at full speed for 15 seconds. The homogenate was centrifuged at 18,000 r.p.m for 15 min at 4° C. in a Sorvall RC5C centrifuge. Supernatant was discarded, and homogenate re-suspended in extraction buffer then centrifugation was repeated. The final pellet was resuspended in 50 mM Trizma pre-set crystals (pH 7.4 @ 37° C.) and stored in 1 ml aliquot tubes at −80° C. (D2=3.0E+08 cells, D3=7.0E+07 cells and D4=1.0E+08 cells). The protein content was determined using a BCA protocol and bovine serum albumin as a standard (Smith, P. K., et al., Measurement of protein using bicinchoninic acid. Anal. Biochem. 150, 76-85 (1985)).

Binding experiments: Crude D2/D3 cell membranes were incubated with 0.03 nM [¹²⁵I]-Iodosulpride (˜2000 Ci/mmol; Amersham, U. K.) and D4 with 0.8 nM [³H]-YM-09151 (˜85 Ci/mmol; NEN, UK), and the test compound in a buffer containing 50 mM Trizma pre-set crystals (pH 7.4 @ 37° C.), 120 mM NaCl, 5 mM KCl, 2 mM CaCl₂, 1 mM MgCl₂, 0.3% (w/v) bovine serum albumin. The total volume is 0.2 ml and incubated in a water bath at 37° C. for 40 minutes. Following incubation, samples were filtered onto GF/B Unifilters using a Can berra Packard Filtermate, and washed four times with ice-cold 50 mM Trizma pre-set crystals (pH 7.4 @ 37° C.). The radioactivity on the filters was measured using a Can berra Packard Topcount Scintillation counter. Non-specific binding was defined with 10 μM SKF-102161 (YM-09151). For competition curves, 10 serial log concentrations of competing cold drug were used (Dilution range: 10 μM-10 pM). Competition curves were analysed using Inflexion, an iterative curve fitting programme in Excel. Results were expressed as pKi values where pKi=−log10[Ki].

The exemplified compounds have pKi values within the range of 7.0-9.5 at the dopamine D3 receptor. pKi results are only estimated to be accurate to about 0.3-0.5.

### Functional Activity at Cloned Dopamine Receptors

The functional activity of compounds at human D2 and human D3 receptors (i.e. agonism or antagonism) may be determined using a Cytosensor Microphysiometer (McConnell H M et al Science 1992 257 1906-1912). In Microphysiometer experiments, cells (hD2_CHO or hD3_CHO) were seeded into 12 mm Transwell inserts (Costar) at 300000 cells/cup in foetal calf serum (FCS)-containing medium. The cells were incubated for 6 h at 37° C. in 5% CO₂, before changing to FCS-free medium. After a further 16-18 h, cups were loaded into the sensor chambers of the Cytosensor Microphysiometer (Molecular Devices) and the chambers perfused with running medium (bicarbonate-free Dulbecco's modified Eagles medium containing 2 mM glutamine and 44 mM NaCl) at a flow rate of 100 ul/min. Each pump cycle lasted 90 s. The pump was on for the first 60 s and the acidification rate determined between 68 and 88 s, using the Cytosoft programme. Test compounds were diluted in running medium. In experiments to determine agonist activity, cells were exposed (4.5 min for hD2, 7.5 min for hD3) to increasing concentrations of putative agonist at half hour intervals. Seven concentrations of the putative agonist were used. Peak acidification rate to each putative agonist concentration was determined and concentration-response curves fitted using Robofit [Tilford, N. S., Bowen, W. P. & Baxter, G. S. Br. J. Pharmacol. (1995), Vol. 115, 160P]. In experiments to determine antagonist potency, cells were treated at 30 min intervals with five pulses of a submaximal concentration of quinpirole (100 nM for hD2 cells, 30 nM for hD3 cells), before exposure to the lowest concentration of putative antagonist. At the end of the next 30 min interval, cells were pulsed again with quinpirole (in the continued presence of the antagonist) before exposure to the next highest antagonist concentration. In all, five concentrations of antagonist were used in each experiment. Peak acidification rate to each agonist concentration was determined and concentration-inhibition curves fitted using Robofit.

The present invention is illustrated using the following examples.

### Preparation 1: 5-{5-[(3-Chloropropyl)thio]-4-methyl-4H-1,2,4-triazol-3-yl}-2-methylquinoline

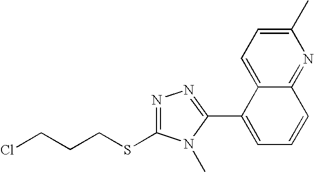

To 4-methyl-5-(2-methyl-5-quinolinyl)-2,4-dihydro-3H-1,2,4-triazole-3-thione (3.6 g, preparation reported in WO 02/40471) in ethanol (60 ml) containing 1-bromo-3-chloropropane (2.0 ml) was carefully added with stirring sodium hydride (0.60 g, 60% in mineral oil). The mixture was heated at reflux for 45 min. Volatiles were evaporated in vacuo and the residue submitted to column chromatography (EtOAc-acetone gradient). The material thus obtained was precipitated from hot EtOAc (20 ml) by adding petroleum ether (40-60, 50 ml), cooled and collected by filtration to provide the title compound as colourless crystals (2.1 g).

NMR (¹H, CDCl₃): δ 8.18 (d, 1H), 8.12 (d, 1H), 7.76 (t, 1H), 7.55 (d, 1H), 7.30 (d, 1H), 3.75 (t, 2H), 3.50 (t, 2H), 3.40 (s, 3H), 2.76 (s, 3H), 2.37 (m, 2H).

### Preparation 2: 3-[(3-Chloropropyl)thio]-4-methyl-5-(4-methyl-1,3-oxazol-5-yl)-4H-1.2.4-triazole

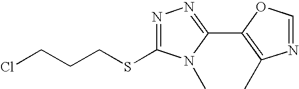

Ethyl 4-methyl-1,3-oxazole-5-carboxylate (7.0 g) was stirred at 25° C. with a solution of sodium hydroxide (8.0 g) in water (70 ml) for 2 h. The resulting solution was cooled in an ice bath and conc. aqueous HCl was slowly added with vigorous stirring until pH 2 had been reached. Filtration, washing with a small volume of cold water and drying resulted in an off-white solid (3.5 g).

This material (5.4 g) was allowed to react in DMF (60 ml) with 4-methyl-3-thiosemicarbazide (4.6 g), 1H-1,2,3-benzotriazol-1-ol (1.1 g), N-[2-(dimethylamino)ethyl]-N′-ethylcarbodiimide hydrochloride (8.6 g), and triethylamine (6.2 ml) for 14 h at 25° C. The solvent was evaporated in vacuo and the residue heated with NaOH (8.5 g) in water (150 ml) at 70° C. for 3.5 h. The resulting solution was cooled in an ice bath and conc. aqueous HCl (17.7 ml) was slowly added with vigorous stirring. Filtration, washing with a small volume of cold water and drying resulted in a yellow powder (5.3 g).

To this material (4.8 g) in EtOH (60 ml) containing 1-bromo-3-chloropropane (3.7 ml) was carefully added with stirring sodium hydride (1.1 g, 60% in mineral oil). The mixture was heated at 60° C. for 1.5 h. Acetic acid (0.15 ml) was added, volatiles evaporated in vacuo and the residue submitted to column chromatography (EtOAc-acetone gradient). The material thus obtained was triturated with cyclohexane to provide the title compound as a faint yellow solid (6.1 g).

NMR (¹H, CDCl₃): δ 7.90 (s, 1H), 3.70 (s, 5H), 3.40 (t, 2H), 2.52 (s, 3H), 2.30 (m, 2H).

### Preparation 3: 1,1-Dimethylethyl 7-hydroxy-1,2,4,5-tetrahydro-benzo[d]azepine-3-carboxylate

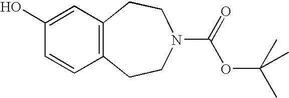

7-Methoxy-2,3,4,5-tetrahydro-1H-3-benzazepine (10 g, preparation reported in WO 02/40471) in 48% aqueous hydrobromic acid (350 ml) was allowed to stir at 10° C. for 4 h. The mixture was allowed to cool to 20° C. then evaporated to dryness, giving the crude hydroxy compound as a brown solid (14.5 g). This solid was dissolved in tetrahydrofuran (100 ml) and water (70 ml) and triethylamine (8 g) was added dropwise, followed by a solution of di-tert-butyl dicarbonate (14 g) in tetrahydrofuran (20 ml). The resulting mixture was allowed to stir at 20° C. for 16 h then partitioned between ethyl acetate (200 ml) and water (200 ml). The aqueous layer was extracted with ethyl acetate (100 ml). The combined organic extracts were washed with saturated aqueous sodium bicarbonate (100 ml), dried over anhydrous sodium sulfate and evaporated to dryness. The resulting oil was purified by chromatography over silica gel, eluting with 10-30% ethyl acetate in hexane, affording the title compound as a white solid (8 g).

NMR (¹H, CD₃OD): δ 6.96 (1H, d), 6.50-6.62 (2H, m), 4.95 (1H, s), 3.40-3.60 (4H, m), 2.75-2.87 (4H, m), 1.48 (9H, s). MS (m/z): 164 [MH-Boc]⁺.

### Preparation 4: 1,1-Dimethylethyl 7-hydroxy-6-nitro-1,2,4,5-tetrahydro-3H-3-benzazepine-3-carboxylate

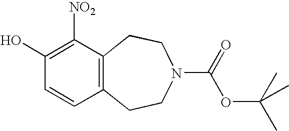

Nitric acid (70%, 3.44 ml) was added dropwise over 40 min with vigorous stirring to 1,1-dimethylethyl 7-hydroxy-1,2,4,5-tetrahydro-benzo[d]azepine-3-carboxylate (14.2 g) in DCM with cooling in an ice bath. After additional 5 min silica gel (15 g) and sodium bicarbonate powder (5 g) were added and stirring was continued for 1.5 h. The mixture was filtered through a layer (1 cm) of silica gel and the solids washed with EtOAc. The resulting solution was concentrated and for separation from 1,1-dimethylethyl 7-hydroxy-8-nitro-1,2,4,5-tetrahydro-3H-3-benzazepine-3-carboxylate submitted to column chromatography to provide the title compound as a yellow solid (5.40 g).

NMR (¹H, CDCl₃): δ 8.55 (s, 1H), 7.20 (d, 1H), 6.90 (d, 1H), 3.65 (m, 2H), 3.52 (m, 2H), 2.85-3.05 (m, 4H), 1.40 (s, 9H); MS (m/z): 307 [M−H]⁻.

### Preparation 5: 1,1-Dimethylethyl 6-amino-7-hydroxy-1,2,4,5-tetrahydro-3H-3-benzazepine-3-carboxylate

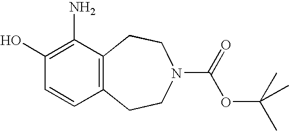

1,1-Dimethylethyl 7-hydroxy-6-nitro-1,2,4,5-tetrahydro-3H-3-benzazepine-3-carboxylate (5.4 g) was hydrogenated for 6 h under atmospheric pressure and 25° C. in the presence of 10% Pd/C (0.9 g) in EtOH (130 ml). The catalyst was removed by filtration and volatiles evaporated to provide the title compound as a faint pink solid (4.7 g).

NMR (¹H, CDCl₃): δ 6.45-6.55 (m, 2H), 4.35 (vbs, 3H), 3.48-3.6 (m, 4H), 2.80 (m, 4H), 1.42 (s, 9H).

### Preparation 6: 1,1-Dimethylethyl 2-methyl-6,7,9,10-tetrahydro-8H-[1,3]oxazolo[4,5-g][3]benzazepine-8-carboxylate

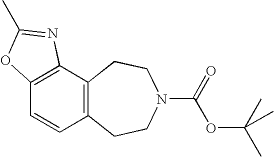

1,1-Dimethylethyl 6-amino-7-hydroxy-1,2,4,5-tetrahydro-3H-3-benzazepine-3-carboxylate (0.34 g) and trimethyl orthoacetate (0.24 ml) in the presence of pyridinium para-toluenesulfonate (0.032 g) were heated in dry DMF (4 ml) for 90 min at 70° C. followed by 2 h at 105° C. Volatiles were evaporated in vacuo to give the title compound which was used without further purification.

MS (m/z): 247 [M−C₄H₈+H]⁺.

### Preparation 7: 2-Methyl-7,8,9,10-tetrahydro-6H-[1,3]oxazolo[4,5-g][3]benzazepine

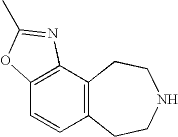

1,1-Dimethylethyl 2-methyl-6,7,9,10-tetrahydro-8H-[1,3]oxazolo[4,5-g][3]benzazepine-8-carboxylate (from Preparation 6) was exposed to trifluoroacetic acid (2 ml) in DCM (2 ml) for 90 min at 25° C. After elimination of volatiles in vacuo the residue was partitioned between aqueous Na₂CO₃ (2 M) and DCM. The organic layer was collected and the aqueous phase extracted twice with DCM. The combined DCM layers were concentrated to give the title compound (0.25 g) as a brown solidified foam.

MS (m/z): 203 [M+H]⁺.

### Preparation 8: 2-Ethyl-7,8,9,10-tetrahydro-6H-[1,3]oxazolo[4,5-g][3]benzazepine

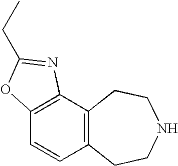

A mixture of 1,1-dimethylethyl 6-amino-7-hydroxy-1,2,4,5-tetrahydro-3H-3-benzazepine-3-carboxylate (500 mg), propionyl chloride (0.173 ml), and pyridine (0.160 ml), in chlorobenzene (4 ml) was stirred at 90° C. for 4 h. Solvent was removed under vacuum, the residue was dissolved in DCM (4 ml) and TFA (10 eq) was added at room temperature. The reaction mixture was stirred until complete deprotection (16 h). Solvent was removed under vacuum, the residue was dissolved in chlorobenzene (8 ml) and stirred at 120° C. for 8 h. The reaction mixture was cooled to room temperature, solvent was removed under vacuum, and the residue was partitioned between aqueous saturated NaHCO₃ and DCM. The organic layer was collected and the aqueous phase extracted twice with DCM. The combined DCM layers were concentrated and submitted to column chromatography to provide the title compound as a yellow liquid (132 mg).

MS (m/z): 217 [MH]⁺.

### Preparation 9: 2-(Trifluoromethyl)-7,8,9,10-tetrahydro-6H-[1,3]oxazolo[4,5-g][3]benzazepine

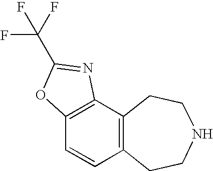

A mixture of 1,1-dimethylethyl 6-amino-7-hydroxy-1,2,4,5-tetrahydro-3H-3-benzazepine-3-carboxylate (0.5 g), trifluoroacetic anhydride (0.28 mL), and pyridine (0.18 mL), in chlorobenzene (4 ml) was stirred at 90° C. for 4 h. The solvent was removed under vacuum, the residue dissolved in DCM (4 ml) and TFA (10 eq.) was added at room temperature. The reaction mixture was stirred until complete deprotection (16 h). Solvent was removed under vacuum, the residue was dissolved in chlorobenzene (8 ml) and stirred at 120° C. for 8 h. The reaction mixture was cooled to room temperature, solvent was removed under vacuum, the residue was partitioned between aqueous saturated NaHCO₃ and DCM. The organic layer was collected and the aqueous phase extracted twice with DCM. The combined DCM layers were concentrated and submitted to column chromatography to provide the title compound as a colorless liquid (0.35 g).

(m/z): 257 [MH]⁺.

### Preparation 10: 3-Acetyl-7-nitro-2,3,4,5-tetrahydro-1H-3-benzazepine

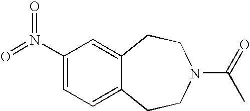

To a cooled solution of HNO₃ (70%) at 10° C., 3-acetyl-2,3,4,5-tetrahydro-1H-3-benzazepine (6.8 g, preparation reported in WO 02/40471) was added portionwise over 20 min. After addition the mixture was allowed to warm to rt. and stirred for a further 4 h. The mixture was poured into ice and the pH adjusted to 11 using NaOH (2N), then extracted with DCM. The organic layer was collected and the aqueous phase extracted twice with DCM. The combined DCM layers were concentrated to provide the target compound (7.7 g), which was used in further conversions without any purification.

NMR (¹H, CDCl₃): δ 8.00 (s, 1H), 7.25 (dd, 2H), 3.75 (m, 2H), 3.60 (m, 2H), 3.05 (2t, 4H), 2.15 (s, 3H); MS (m/z): 235 [MH]⁺.

### Preparation 11: 7-Nitro-2,3,4,5-tetrahydro-1H-3-benzazepine

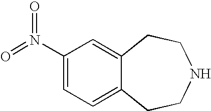

A solution of 3-acetyl-7-nitro-2,3,4,5-tetrahydro-1H-3-benzazepine (2.9 g) in HCl (conc., 200 ml) was heated at reflux for 48 h. The mixture was cooled at 0° C. and neutralised with NaOH (conc.), then extracted with DCM. The organic layer was collected and the aqueous phase extracted twice with DCM. The combined DCM layers were concentrated to provide the target compound (2.3 g).

MS (m/z): 193 [MH]⁺.

### Preparation 12: 1,1-Dimethylethyl 7-nitro-1,2,4,5-tetrahydro-3H-3-benzazepine-3-carboxylate

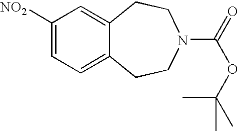

To a solution of 7-nitro-2,3,4,5-tetrahydro-1H-3-benzazepine (2.33 g) in dry DCM (120 ml), triethylamine (4.2 ml, 2.5 eq), bis(1,1-dimethylethyl) dicarbonate (6.1 g, 2.3 eq.), and N,N-dimethyl-4-pyridinamine (296 mg) were added. The mixture was stirred at rt. for 1 h. Solvent was evaporated in vacuo and the material thus obtained was purified by flash chromatography over silica gel, eluting with 20% ethyl acetate in hexane, affording the title compound (3.25 g).

MS (m/z): 237 [M−56]⁺.

### Preparation 13: 1,1-Dimethylethyl 7-amino-1,2,4,5-tetrahydro-3H-3-benzazepine-3-carboxylate

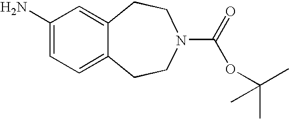

1,1-Dimethylethyl 7-nitro-1,2,4,5-tetrahydro-3H-3-benzazepine-3-carboxylate (3.25 g), was hydrogenated for 3 h under atmospheric pressure at 25° C. in the presence of 10% Pd/C (0.32 g) in MeOH (130 ml). The catalyst was removed by filtration and volatiles evaporated and the resulting residue was purified by flash chromatography over silica gel, eluting with 40% ethyl acetate in hexane, affording the title compound (2.55 g).

MS (m/z): 207 [M−56]⁺.

### Preparation 14: 1,1-Dimethylethyl 7-(acetylamino)-1,2,4,5-tetrahydro-3H-3-benzazepine-3-carboxylate

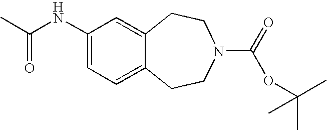

1,1-Dimethylethyl 7-amino-1,2,4,5-tetrahydro-3H-3-benzazepine-3-carboxylate (995 mg) was stirred in dry DCM (10 ml) and treated with triethylamine (0.634 ml) and acetyl chloride (0.275 ml) and stirred at rt. for 3 h. The mixture was then partitioned between aqueous saturated NaHCO₃ and DCM. The organic layer was collected and the aqueous phase extracted twice with DCM. The combined DCM layers were concentrated and the residue was purified by flash chromatography over silica gel, eluting with 50% ethyl acetate in hexane, affording the title compound (818 mg).

MS (m/z): 305 [MH]⁺, 327 [M+Na]⁺.

### Preparation 15: 1,1-Dimethylethyl 7-(ethanethioylamino)-1,2,4,5-tetrahydro-3H-3-benzazepine-3-carboxylate

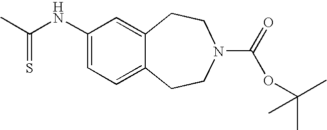

1,1-Dimethylethyl-7-(acetylamino)-1,2,4,5-tetrahydro-3H-3-benzazepine-3-carboxylate (818 mg) and Lawesson's reagent (1.1 g) were combined in toluene (10 ml) and refluxed for 1.5 h. After cooling the reaction was partitioned between ethyl acetate and saturated NaHCO₃. The combined organic layers were concentrated and the residue was purified by flash chromatography over silica gel, eluting with 40% ethyl acetate in hexane, affording the title compound (411 mg).

MS (m/z): 321 [MH]⁺, 265 [M−56]⁺.

### Preparation 16: 1,1-Dimethylethyl 2-methyl-6,7,9,10-tetrahydro-8H-[1,3]thiazolo[5,4-g][3]benzazepine-8-carboxylate

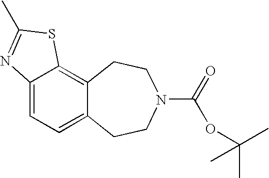

1,1-Dimethylethyl 7-(ethanethioylamino)-1,2,4,5-tetrahydro-3H-3-benzazepine-3-carboxylate (411 mg), was dissolved in MeOH (12 ml), and 1 M NaOH (6.1 ml) and added to potassium ferricyanide [K₃Fe(CN)₆] (2.1 g), in H₂O (12 ml). This mixture was warmed up to 60° C. for 1.5 h, cooled to rt., concentrated and the material thus obtained was purified by flash chromatography over silica gel, eluting with 20% ethyl acetate in hexane, affording the title compound (167 mg).

MS (m/z): 321 [MH]⁺, 265 [M−56]⁺.

### Preparation 17: 2-Methyl-7,8,9,10-tetrahydro-6H-[1,3]thiazolo[5,4-g][3]benzazepine

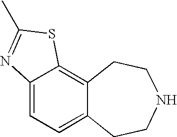

1,1-Dimethylethyl-2-methyl-6,7,9,10-tetrahydro-8H-[1,3]thiazolo[5,4-g][3]benzazepine-8-carboxylate (167 mg) was dissolved in DCM (5 mL) and added to TFA (2 mL). The mixture was stirred at rt for 1 h and then concentrated to afford the title compound (139 mg) which was submitted to the next reaction without any further purification.

MS (m/z): 219 [MH]⁺.

### Preparation 18: 2-(1,3-Dimethyl-1H-pyrazol-5-yl)-7,8,9,10-tetrahydro-6H-[1,3]oxazolo[4,5-g][3]benzazepine

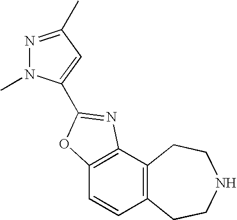

The title compound was prepared as described in General Procedure 2 from 1,1-dimethylethyl 6-amino-7-hydroxy-1,2,4,5-tetrahydro-3H-3-benzazepine-3-carboxylate and 1,3-dimethyl-1H-pyrazole-5-carbonyl chloride. The latter reactant had been obtained in dichloromethane solution from the reaction of 1,3-dimethyl-1H-pyrazole-5-carboxylic acid (1.3 eq.) in the presence of a catalytic quantity of dimethylformamide, followed by evaporation of the volatiles after 1.5 h at 25° C.

MS (m/z): 283 [MH]⁺.

### Preparation 19: 2-(Pentafluoroethyl)-7,8,9,10-tetrahydro-6H-[1,3]oxazolo[4,5-g][3]benzazepine

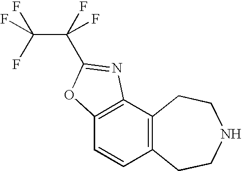

1,1-Dimethylethyl 6-amino-7-hydroxy-1,2,4,5-tetrahydro-3H-3-benzazepine-3-carboxylate (0.28 g) was treated with pentafluoropropionyl anhydride (1.2 eq.) and pyridine (1.2 eq.) in chlorobenzene at reflux for 5 h. Volatiles were evaporated, aqueous Na₂CO₃ carefully added until ca. pH 8-9 had been reached, and the mixture extracted with dichloromethane.

The title compound (0.29 g) was obtained as a colourless solid following evaporation of the volatiles and column chromatography. The material thus obtained was directly used without further characterisation.

### Preparation 20: 1,1-Dimethylethyl 6-hydroxy-1,2,4,5-tetrahydro-3H-3-benzazepine-3-carboxylate

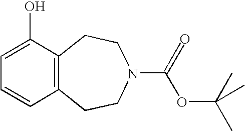

To a solution of hydroxylamine hydrochloride (49.29 g) and sodium acetate (93.09 g) in water (260 mL) a solution of 8-(methyloxy)-3,4-dihydro-2(1H)-naphthalenone (25 g) in ethanol (700 mL) was added over 45 min. The formation of white solid was observed. The mixture was stirred for 10 min, then most ethanol evaporated in vacuo and the aqueous phase extracted with dichloromethane (3×400 mL). The collected organic phases were washed with a saturated solution of sodium bicarbonate (500 mL), water (500 mL), then dried over sodium sulphate and concentrated in vacuo to give 26 g of 8-(methyloxy)-3,4-dihydro-2(1H)-naphthalenone oxime as a mixture of isomers as a white solid (MS (m/z): 192 [MH]⁺).

To a solution of material thus obtained (27 g) in acetone (400 mL) a solution of sodium carbonate (67.34 g) in water (600 mL) was added at room temperature. Then a solution of 4-methylbenzenesulfonyl chloride (40.83 g) in acetone (200 mL) was added dropwise over 30 min. Formation of white solid was observed. The mixture was stirred at room temperature for 1 h, at reflux for 2 h, then again at room temperature overnight and once more at reflux for 1 h. Most acetone was evaporated in vacuo and the residual mixture extracted with dichloromethane (3×500 mL). The collected organic phases were washed with water (500 mL), then dried over sodium sulphate and concentrated in vacuo. The residue was submitted to column chromatography (eluent AcOEt) to give 9-(methyloxy)-1,3,4,5-tetrahydro-2H-3-benzazepin-2-one containing a minor isomer (14.9 g).

To a solution of the material thus obtained (14.9 g) in THF (160 mL) borane (1M in THF, 234 mL) was added dropwise at 0° C. under nitrogen. The mixture was stirred at reflux for 3 h, kept at room temperature for 64 h and heated once more at reflux for 2 h. With cooling at 0° C. HCl (6 M, 200 mL) was carefully added dropwise (gas evolution and exothermic process). The mixture was stirred at room temperature for 16 h, then NaOH (6 M) was added dropwise until pH=9. The mixture was diluted with water and extracted with ethyl acetate, the organic phase washed with brine, then dried over sodium sulphate and concentrated in vacuo to give 13.3 g of 6-(methyloxy)-2,3,4,5-tetrahydro-1H-3-benzazepine containing a minor isomer.

To a solution of the crude material thus prepared (13.3 g) in dichloromethane (490 mL) at −78° C. was added dropwise under nitrogen BBr₃ (1 M in dichloromethane, 150 mL). The mixture was stirred at room temperature for 3 h, then aqueous Na₂CO₃ (2 M) was added at 0° C. until pH=9.

The organic phase was evaporated in vacuo. The residual water was diluted with THF (400 mL) and bis(1,1-dimethylethyl) dicarbonate (14.73 g) was added portionwise. The reaction mixture was stirred at room temperature for 90 min. Organic solvent was removed in vacuo, residual water acidified to pH=6 with HCl (1 M) and extracted with dichloromethane. The organic phase was dried over sodium sulphate and concentrated in vacuo. The crude material was purified by flash chromatography (Cyclohexane/AcOEt 8:2) to give 12.1 g of the title compound as a white solid.

NMR (¹H, CDCl₃): δ 7.00 (t, 1H), 6.74 (d, 1H), 6.68 (d, 1H), 5.00 (bs, 1H), 3.58 (m, 4H), 3.02 (m, 2H), 2.92 (m, 2H), 1.51 (s, 9H).

### Preparation 21: 1,1-Dimethylethyl 6-hydroxy-7-nitro-1,2,4,5-tetrahydro-3H-3-benzazepine-3-carboxylate

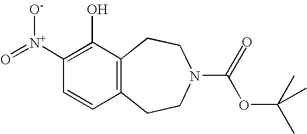

Nitric acid (70%, 0.37 mL) was added dropwise over 15 min with vigorous stirring to 1,1-dimethylethyl 6-hydroxy-1,2,4,5-tetrahydro-3H-3-benzazepine-3-carboxylate (1.5 g) in dichloromethane (10 mL) containing silica gel 60 (4.5 g), with cooling in a cold bath at −15° C. After 1 h the mixture was allowed to warm to 25° C., sodium bicarbonate powder (0.5 g) was added and stirring was continued for 16 h. The mixture was filtered and the solids washed with EtOAc. The resulting solution was concentrated and submitted to column chromatography to provide the title compound as a yellow solid (0.71 g).

NMR (¹H, CDCl₃): δ 11.16 (s, 1H), 7.94 (d, 1H), 6.79 (d, 1H), 3.6 (m, 4H), 3.13 (m, 2H), 3.0 (m, 2H), 1.50 (s, 9H). MS (m/z): 253 [M−C₄H₈+H]⁺, 331 [MNa]⁺.

### Preparation 22: 1,1-Dimethylethyl 7-amino-6-hydroxy-1,2,4,5-tetrahydro-3H-3-benzazepine-3-carboxylate

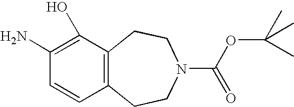

1,1-Dimethylethyl 6-hydroxy-7-nitro-1,2,4,5-tetrahydro-3H-3-benzazepine-3-carboxylate (0.70 g) was hydrogenated for 6 h under atmospheric pressure and 25° C. in the presence of 10% Pd/C (0.11 g) in EtOH (5 mL) and EtOAc (10 mL). The catalyst was removed by filtration and volatiles evaporated to provide the title compound as a faint purple solid (0.65 g).

NMR (¹H, CDCl₃): δ 6.6 (d, 1H), 6.53 (d, 1H), 3.1-3.6 (brm, 6H), 2.9-2.97 (m, 2H), 2.75-2.82 (m, 2H), 1.42 (s, 9H), one acidic proton (OH) not observed.

### Preparation 23: 2-(1,1-Difluoroethyl)-7,8,9,10-tetrahydro-6H-[1,3]oxazolo[5,4-g][3]benzazepine

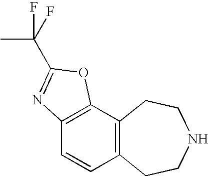

2,2-Difluoropropionic acid (0.28 g) was allowed to react in chlorobenzene (4.7 mL) containing 3 drops dimethylformamide with oxalyl chloride (0.22 g) for 1.5 h at 25° C., resulting in a ca. 0.5 M solution. 1.6 mL of this solution was added to 1,1-dimethylethyl 7-amino-6-hydroxy-1,2,4,5-tetrahydro-3H-3-benzazepine-3-carboxylate (0.17 g) containing pyridine (0.13 mL) and heated at 110° C. for 2 h. Polyphosphoric acid (ca. 0.4 g) was added and heating continued at 110° C. for 40 min. Volatiles were evaporated, to the residue with cooling in an ice bath carefully added water and Na₂CO₃ (until pH 8-9) and the mixture extracted 3 times with dichloromethane. The title compound (0.084 g) was obtained as a faint yellow oil following evaporation of the volatiles from the combined organic extracts and used in the next step without further purification and characterisation.

### Preparation 24: 8-(3-chloropropyl)-2-(trifluoromethyl)-7,8,9,10-tetrahydro-6H-[1,3]oxazolo[4,5-g][3]benzazepine

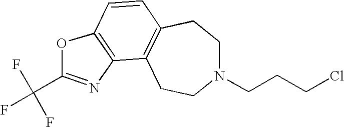

To a solution of 2-(trifluoromethyl)-7,8,9,10-tetrahydro-6H-[1,3]oxazolo[4,5-g][3]benzazepine (0.19 g) in dry THF (5 ml), diisopropylethylamine (0.39 ml) and 1-bromo-3-chloropropane (0.74 mL) were added and the resulting mixture was refluxed for 7 hours. After cooling at room temperature it was diluted with ethyl acetate (20 ml) washed twice with a saturated solution of NaHCO₃ in water (15 ml), dried over Na₂SO₄ and concentrated under reduced pressure. The crude was purified by flash chromatography eluting with cHex/EtOAc 8:2 to give the title compound as a colourless oil (0.20 g). MS (m/z): 333[MH]⁺.

### Preparation 25: 1,1-dimethylethyl 7-(propanoylamino)-1,2,4,5-tetrahydro-3H-3-benzazepine-3-carboxylate

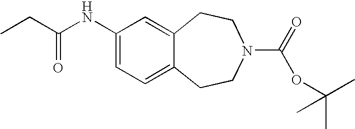

1,1-Dimethylethyl 7-amino-1,2,4,5-tetrahydro-3H-3-benzazepine-3-carboxylate (1.5 g) was stirred in dry DCM (15 ml) and treated with TEA (0.957 ml) and propanoyl chloride (0.510 ml) and stirred at rt. for 1 h. Mixture was then partitioned between aqueous saturated NaHCO₃ and DCM. The organic layer was collected and the aqueous phase extracted twice with DCM. The combined DCM layers were concentrated and the residue was purified by flash chromatography over silica gel, eluting with 50% ethyl acetate in hexane, affording the title compound (1.26 g).

MS (m/z): 318 [MH]⁺

### Preparation 26: 1,1-dimethylethyl 7-(propanethioylamino)-1,2,4,5-tetrahydro-3H-3-benzazepine-3-carboxylate

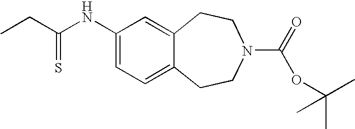

1,1-Dimethylethyl 7-(propanoylamino)-1,2,4,5-tetrahydro-3H-3-benzazepine-3-carboxylate (1.26 mg) and Lawesson's reagent (1.6 g) were combined in toluene (20 ml) and refluxed for 1.5 h. After cooling the reaction was worked up with ethyl acetate/NaHCO₃ (sat. sol.). The combined organic layers were concentrated and the residue was purified by flash chromatography over silica gel, eluting with 40% ethyl acetate in hexane, affording the title compound (750 mg).

MS (m/z): 334 [MH]⁺.

### Preparation 27: 1,1-dimethylethyl 2-ethyl-6,7,9,10-tetrahydro-8H-[1,3]thiazolo[5,4-g][3]benzazepine-8-carboxylate

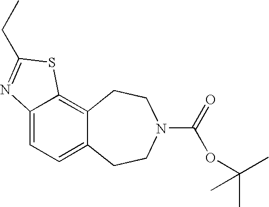

1,1-Dimethylethyl 7-(propanethioylamino)-1,2,4,5-tetrahydro-3H-3-benzazepine-3-carboxylate, was dissolved in MeOH (22 ml), and 1N NaOH (11 ml) and added to potassium ferricyanide [K₃Fe(CN)₆] (3.7 g), in H₂O (22 ml). This mixture was warmed up to 60° C. for 1.5 h, cooled at rt. concentrated and the material thus obtained was purified by flash chromatography over silica gel, eluting with 20% ethyl acetate in hexane, affording a mixture (2/1) of title compound and its regioisomer (653 mg).

MS (m/z): 332 [MH]⁺.

### Preparation 28: 2-ethyl-7,8,9,10-tetrahydro-6H-[1,3]thiazolo[5,4-g][3]benzazepine

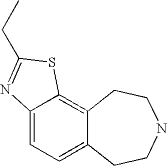

1,1-dimethylethyl 2-ethyl-6,7,9,10-tetrahydro-8H-[1,3]thiazolo[5,4-g][3]benzazepine-8-carboxylate (653 mg), was dissolved in DCM (20 ml) and added to TFA (4 ml). Mixture was stirred at rt. for 1 h and then concentrated affording a mixture (2/1) of title compound and its regioisomer (422 mg), which was submitted to the next reaction without any further purification.

MS (m/z): 232 [MH]⁺

### General Procedure 1: N-alkylation and Salt Formation

A mixture of the secondary amine (1 eq.), the primary chloroalkyl derivative (1.2 eq.), sodium iodide (1 eq.), potassium carbonate (1.2 eq.) in dry DMF (2 ml per mmol secondary amine) was stirred under vacuum to remove traces of humidity and undesired solvent residues, then purged with dry nitrogen. The mixture was stirred at 60° C. for 24 h. After elimination of the solvent in vacuo the residue was partitioned between aqueous Na₂CO₃ (0.5 M) and DCM. The organic layer was collected and the aqueous phase extracted twice with DCM. The combined DCM layers were concentrated and submitted to column chromatography to provide the free base of the target compounds. To a solution of this material in DCM (ca. 20 ml per mmol) was added HCl in Et₂O (1.0 eq. with respect to free base), the solvent evaporated in vacuo and the material thus obtained was triturated with 1:1 Et₂O:EtOAc (ca. 10 ml per mmol) to give the target compounds as hydrochloride salts.

General Procedure 2: Synthesis of 2-Substituted 6,7,8,9-tetrahydro-5H-[1,3]oxazolo[4,5-g][3]benzazepines

A mixture of 1,1-dimethylethyl 7-amino-8-hydroxy-1,2,4,5-tetrahydro-3H-3-benzazepine-3-carboxylate (1 eq.), a carboxylic acid chloride derivative (1.1 eq.), and pyridine (1.1 eq.), in chlorobenzene (2 ml per mmol of benzazepine derivative) was stirred at 90° C. for 4 h. Solvent was removed under vacuum, the residue dissolved in DCM (2 ml per mmol of benzazepine derivative) and trifluoroacetic acid (10 eq.) was added at room temperature. The reaction mixture was stirred until complete deprotection (4-16 h). Solvent was removed under vacuum, the residue was dissolved in chlorobenzene (4 ml per mmol of benzazepine derivative) and stirred at 120° C. for 8 h. The reaction mixture was cooled to room temperature, solvent was removed under vacuum, the residue was partitioned between aqueous saturated NaHCO₃ and DCM. The organic layer was collected and the aqueous phase extracted twice with DCM. The combined DCM layers were concentrated and submitted to column chromatography to provide the free base of the target compounds which were used in further conversions.

### EXAMPLE 1

### 2-Methyl-8-(3-{[4-methyl-5-(2-methyl-5-quinolinyl)-4H-1,2,4-triazol-3-yl]thio}propyl)-7,8,9,10-tetrahydro-6H-[1,3]oxazolo[4,5-g][3]benzazepine hydrochloride

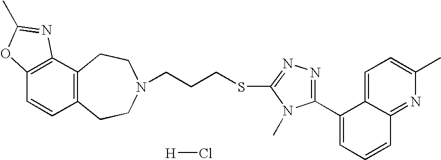

The title compound was prepared in analogy to General Procedure 1 from 2-methyl-7,8,9,10-tetrahydro-6H-[1,3]oxazolo[4,5-g][3]benzazepine (0.30 mmol) and 5-{5-[(3-chloropropyl)thio]-4-methyl-4H-1,2,4-triazol-3-yl}-2-methylquinoline to give the title compound (0.18 mmol) as a colourless slightly hygroscopic solid.

NMR (¹H, CD₃OD): δ 8.37 (bd, 1H), 8.27 (d, 1H), 8.03 (dd, 1H), 7.88 (d, 1H), 7.66 (d, 1H), 7.43 (d, 1H), 7.27 (d, 1H), 3.8-4.0 (bm, 3H), 3.57 (s, 3H), 3.47-3.53 (2t, 4H), 3.1-3.6 (bm, 5H), 2.85 (s, 3H), 2.66 (s, 3H), 2.43 (m, 2H), acidic proton not observed. MS (m/z): 499 [MH]⁺.

### EXAMPLE 2

### 2-Ethyl-8-(3-{[4-methyl-5-(2-methyl-5-quinolinyl)-4H-1,2,4-triazol-3-yl]thio}propyl)-7,8,9,10-tetrahydro-6H-[1,3]oxazolo[4,5-g][3]benzazepine hydrochloride

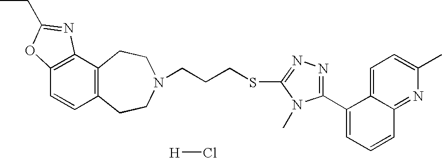

The title compound was prepared in analogy to General Procedure 1 from 2-ethyl-7,8,9,10-tetrahydro-6H-[1,3]oxazolo[4,5-g][3]benzazepine (73 mg) and 5-{5-[(3-chloropropyl)thio]-4-methyl-4H-1,2,4-triazol-3-yl}-2-methylquinoline (108 mg) to give the title compound (91 mg) as a white slightly hygroscopic solid.

NMR (1H, CD₃OD): δ 9.07 (d, 1H), 8.41 (d, 1H), 8.32 (t, 1H), 8.19 (dd, 1H), 8.03 (d, 1H), 7.43 (d, 1H), 7.27 (d, 1H), 4-3.1 (m, 12H), 3.66 (s, 3H), 3.07 (s, 3H), 3 (q, 2H), 2.45 (m, 2H), 1.45 (t, 3H); acidic proton not observed. MS (m/z): 513 [MH]⁺.

### EXAMPLE 3

### 2-Ethyl-8-(3-{[4-methyl-5-(4-methyl-1,3-oxazol-5-yl)-4H-1,2,4-triazol-3-yl]thio}propyl)-7,8,9,10-tetrahydro-6H-[1,3]oxazolo[4,5-g][3]benzazepine hydrochloride

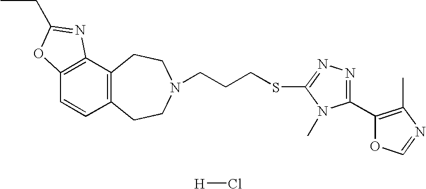

The title compound was prepared in analogy to General Procedure 1 from 2-ethyl-7,8,9,10-tetrahydro-6H-[1,3]oxazolo[4,5-g][3]benzazepine (46 mg) and 3-[(3-chloropropyl)thio]-4-methyl-5-(4-methyl-1,3-oxazol-5-yl)-4H-1,2,4-triazole (69 mg) to give the title compound (25 mg) as a white slightly hygroscopic solid.

NMR (¹H, CD₃OD): δ 8.27 (s, 1H), 7.36 (d, 1H), 7.13 (d, 1H), 4.49 (m, 8H), 3.69 (s, 3H), 3.26 (m, 4H), 2.88 (q, 2H), 2.34 (s, 3H), 2.20 (m, 2H), 1.33 (t, 3H); acidic proton not observed. MS (m/z): 453 [MH]⁺.

### EXAMPLE 4

### 8-(3-{[4-Methyl-5-(2-methyl-5-quinolinyl)-4H-1,2,4-triazol-3-yl]thio}propyl)-2-(trifluoromethyl)-7,8,9,10-tetrahydro-6H-[1,3]oxazolo[4,5-g][3]benzazepine hydrochloride

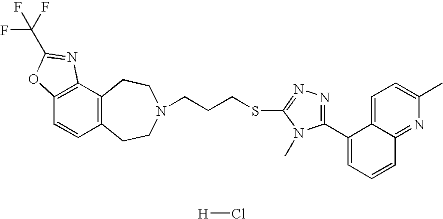

The title compound was prepared in analogy to General Procedure 1 from 2-(trifluoromethyl)-7,8,9,10-tetrahydro-6H-[1,3]oxazolo[4,5-g][3]benzazepine (0.4 mmol) and 5-{5-[(3-chloropropyl)thio]-4-methyl-4H-1,2,4-triazol-3-yl}-2-methylquinoline to give the title compound (0.12 mmol) as a white slightly hygroscopic solid.

NMR (¹H, CD₃OD): δ 9.07 (d, 1H), 8.39 (d, 1H), 8.30 (t, 1H), 8.18 (d, 1H), 8.02 (d, 1H), 7.66 (dd, 1H), 7.52 (dd, 1H), 4-3.2 (m, 8H), 3.64 (s, 3H), 3.5 (m, 4H), 3.05 (s, 3H), 2.44 (m, 2H); acidic proton not observed. MS (m/z): 553 [MH]⁺.

### EXAMPLE 5

### 8-(3-{[4-Methyl-5-(4-methyl-1,3-oxazol-5-yl)-4H-1,2,4-triazol-3-yl]thio}propyl)-2-(trifluoromethyl)-7,8,9,10-tetrahydro-6H-[1,3]oxazolo[4,5-g][3]benzazepine hydrochloride

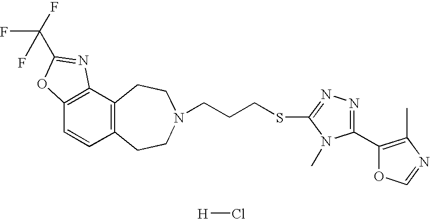

The title compound was prepared in analogy to General Procedure 1 from 2-(trifluoromethyl)-7,8,9,10-tetrahydro-6H-[1,3]oxazolo[4,5-g][3]benzazepine (0.2 mmol) and 3-[(3-chloropropyl)thio]-4-methyl-5-(4-methyl-1,3-oxazol-5-yl)-4H-1,2,4-triazole to give the title compound (0.09 mmol) as a white slightly hygroscopic solid.

NMR (¹H, DMSO): δ 10.5 (bs, 1H), 8.58 (s, 1H), 7.80 (d, 1H), 7.55 (d, 1H), 3.79 (m, 2H), 3.70 (s, 3H), 3.5-3.30 (m, 6H), 3.28 (m, 4H), 2.38 (s, 3H), 2.2 (m, 2H). MS (m/z): 493 [MH]⁺.

### EXAMPLES 6-14

The following examples were prepared in analogy to General Procedure 1 from the corresponding secondary amines and chloropropyl derivatives:

| | | |
|---|---|---|
| 6 | 2-(1,3-Dimethyl-1H-pyrazol-5-yl)-8-(3-{[4- | NMR (¹H, CD₃OD): δ 8.37 (d, |
| | methyl-5-(2-methyl-5-quinolinyl)-4H-1,2,4- | 1 H), 8.27 (d, 1 H), 8.03 (t, |
| | triazol-3-yl]thio}propyl)-7,8,9,10-tetrahydro- | 1 H), 7.88 (d, 1 H), 7.65 (d, |
| | 6H-[1,3]oxazolo[4,5-g][3]benzazepine | 1 H), 7.54 (d, 1 H), 7.37 (d, |
| | hydrochloride | 1 H), 6.89 (s, 1 H), 4.33 (s, |
| | 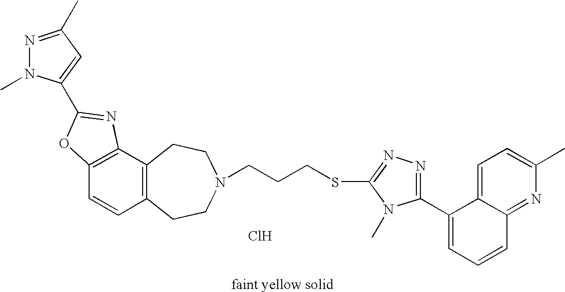 | 3 H), 3.95-4.05 (broad, 4 H),3.57 (s, 3 H), 3.2-3.6 (multiplem, 4 H), 3.56 (t, 2 H), 3.48 (t,2 H), 2.85 (s, 3 H), 2.45 (m,2 H), 2.33 (s, 3 H), acidicproton not observed. MS(m/z): 579 [MH]⁺, 290[M + 2 H]²⁺. |
| 7 | 2-(1,3-Dimethyl-1H-pyrazol-5-yl)-8-(3-{[4- | NMR (¹H, CD₃OD): δ 8.4 (s, |
| | methyl-5-(4-methyl-1,3-oxazol-5-yl)-4H-1,2,4- | 1 H), 7.54 (d, 1 H), 7.36 (d, |
| | triazol-3-yl]thio}propyl)-7,8,9,10-tetrahydro- | 1 H), 6.9 (s, 1 H), 4.33 (s, 3 H), |
| | 6H-[1,3]oxazolo[4,5-g][3]benzazepine | 4.05-3.8 (m, 2 H), 3.83 (s, |
| | hydrochloride | 3 H), 3.5-3.2 (multiple m, 6 H), |
| | 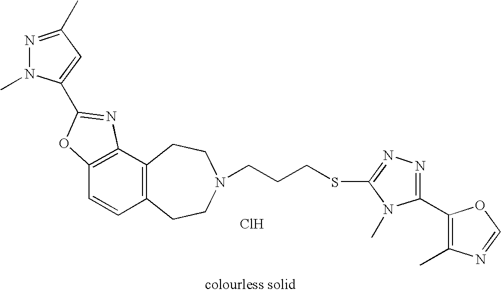 | 3.5 (t, 2 H), 3.41 (t, 2 H), 2.47(s, 3 H), 2.36 (m, 2 H), 2.33 (s,3 H), acidic proton notobserved. MS (m/z): 519[MH]⁺. |
| 8 | 8-(3-{[4-Methyl-5-(2-methyl-5-quinolinyl)-4H- | NMR (¹H, CD₃OD): δ 8.37 (d, |
| | 1,2,4-triazol-3-yl]thio}propyl)-2- | 1 H), 8.27 (d, 1 H), 8.03 (t, |
| | (pentafluoroethyl)-7,8,9,10-tetrahydro-6H- | 1 H), 7.88 (d, 1 H), 7.7 (d, 1 H), |
| | [1,3]oxazolo[4,5-g][3]benzazepine | 7.65 (d, 1 H), 7.57 (d, 1 H), |
| | hydrochloride | 4.05-3.95 (broad, 4 H), 3.57 |
| | 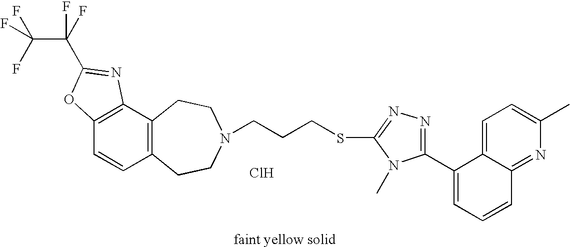 | (s, 3 H), 3.6-3.2 (multiple m,4 H), 3.55 (t, 2 H), 3.46 (t, 2 H),2.85 (s, 3 H), 2.45 (m, 2 H),acidic proton not observed.MS (m/z): 603 [MH]⁺. |
| 9 | 8-(3-{[4-Methyl-5-(4-methyl-1,3-oxazol-5-yl)- | NMR (¹H, CD₃OD): δ 8.4 (s, |
| | 4H-1,2,4-triazol-3-yl]thio}propyl)-2- | 1 H), 7.7 (d, 1 H), 7.56 (d, 1 H), |
| | (pentafluoroethyl)-7,8,9,10-tetrahydro-6H- | 4.05-3.95 (broad, 4 H), 3.82 |
| | [1,3]oxazolo[4,5-g][3]benzazepine | (s, 3 H), 3.6-3.2 (multiple m, |
| | hydrochloride | 4 H), 3.47 (t, 2 H), 3.4 (t, 2 H), |
| | 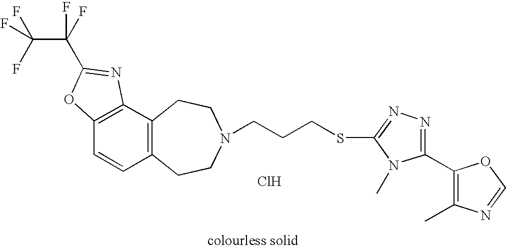 | 2.47 (s, 3 H), 2.37 (m, 2 H),acidic proton not observed.MS (m/z): 543 [MH]⁺. |
| 10 | 8-(3-{[4-Methyl-5-(2-methyl-5-quinolinyl)-4H- | NMR (¹H, CDCl₃): δ 8.22 (d, |
| | 1,2,4-triazol-3-yl]thio}propyl)-2- | 1 H), 8.16 (d, 1 H), 7.82 (t, |
| | (trifluoromethyl)-7,8,9,10-tetrahydro-6H- | 1 H), 7.6 (2 d, 2 H), 7.36 (d, |
| | [1,3]oxazolo[5,4-g][3]benzazepine | 1 H), 7.26 (d, 1 H), 3.51 (t, |
| | 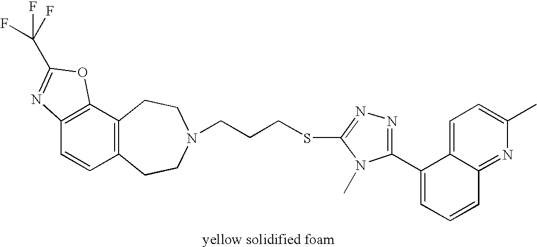 | 2 H), 3.45 (s, 3 H), 3.28 (m,2 H), 3.14 (m, 2 H), 2.8 (s, 3 H),2.7 (m, 6 H), 2.15 (quint., 2 H).MS (m/z): 553 [MH]⁺. |
| 11 | 8-(3-{[4-Methyl-5-(4-methyl-1,3-oxazol-5-yl)- | NMR (¹H, CDCl₃): δ 7.96 (s, |
| | 4H-1,2,4-triazol-3-yl]thio}propyl)-2- | 1 H), 7.6 (d, 1 H), 7.24 (d, 1 H), |
| | (trifluoromethyl)-7,8,9,10-tetrahydro-6H- | 3.74 (s, 3 H), 3.41 (t, 2 H), |
| | [1,3]oxazolo[5,4-g][3]benzazepine | 3.25 (m, 2 H), 3.11 (m, 2 H), |
| | 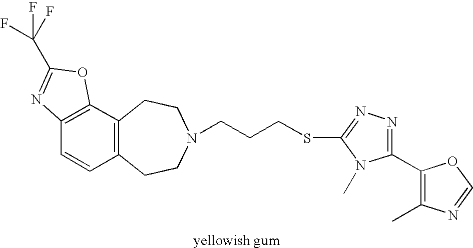 | 2.8-2.7 (m, 6 H), 2.56 (s, 3 H),2.08 (quint., 2 H). |
| 12 | 2-(1,1-Difluoroethyl)-8-(3-{[4-methyl-5-(4- | NMR (¹H, CD₃OD): δ 8.27 (s, |
| | methyl-1,3-oxazol-5-yl)-4H-1,2,4-triazol-3- | 1 H), 7.32 (d, 1 H), 7.19 (d, |
| | yl]thio}propyl)-7,8,9,10-tetrahydro-6H- | 1 H), 3.70 (s, 3 H), 3.18-3.32 |
| | [1,3]oxazolo[4,5-g][3]benzazepine | (m, 4 H), 2.95 (m, 2 H), 2.62 |
| | 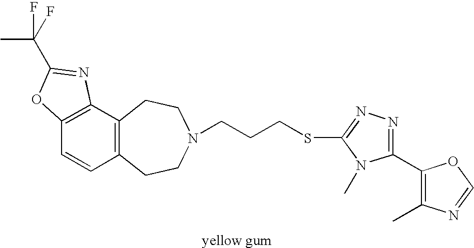 | (m, 6 H), 2.30 (s, 3 H), 2.07 (t,3 H), 1.90 (m, 2 H). MS (m/z):489 [MH]⁺. |
| 13 | 2-(1,1-Difluoroethyl)-8-(3-{[4-methyl-5-(2- | NMR (¹H, CD₃OD): δ 8.36 |
| | methyl-5-quinolinyl)-4H-1,2,4-triazol-3- | (bd, 1 H), 8.14 (d, 1 H), 8.01 (t, |
| | yl]thio}propyl)-7,8,9,10-tetrahydro-6H- | 1 H), 7.86 (d, 1 H), 7.6-7.7 (m, |
| | [1,3]oxazolo[5,4-g][3]benzazepine | 2 H), 7.40 (d, 1 H), 3.85-4.05 |
| | hydrochloride | (bm, 2 H), 3.7-3.8 (bm, 1 H), |
| | 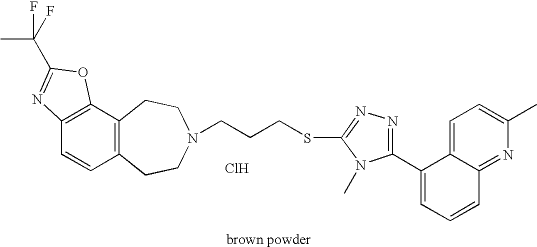 | 3.2-3.6 (multiple m, 12 H),2.83 (s, 3 H), 2.42 (m, 2 H),,2.18 (t, 3 H), acidic proton notobserved. MS (m/z): 549[MH]⁺. |
| 14 | 2-(1,1-Difluoroethyl)-8-(3-{[4-methyl-5-(4- | NMR (¹H, CD₃OD): δ 8.38 (s, |
| | methyl-1,3-oxazol-5-yl)-4H-1,2,4-triazol-3- | 1 H), 7.66 (d, 1 H), 7.39 (d, |
| | yl]thio}propyl)-7,8,9,10-tetrahydro-6H- | 1 H), 3.80 (s, 3 H), 3.47 (t, 2 H), |
| | [1,3]oxazolo[5,4-g][3]benzazepine | 3.37 (t, 2 H), 3.0-4.0 (m, 8 H), |
| | hydrochloride | 2.44 (s, 3 H), 2.34 (m, 2 H), |
| | 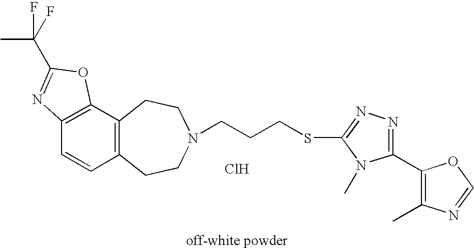 | 2.19 (t, 3 H), acidic proton notobserved. MS (m/z): 489[MH]⁺. |

### EXAMPLE 15

### 8-(3-{[4-methyl-5-(5-methyl-2-pyrazinyl)-4H-1,2,4-triazol-3-yl]thio}propyl)-2-(trifluoromethyl)-7,8,9,10-tetrahydro-6H-[1,3]oxazolo[4,5-g][3]benzazepine

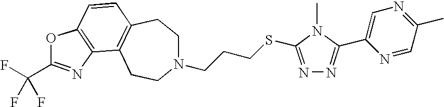

To a solution of 4-methyl-5-(5-methyl-2-pyrazinyl)-2,4-dihydro-3H-1,2,4-triazole-3-thione (25 mg) in dry acetonitrile (1 ml) 2-tert-butylimino-2-diethylamino-1,3-dimethyl-perhydro-

1,3,2-diaza-phosphorine on polystyrene (81 mg) was added and the resulting mixture was shaken for 30 minutes at 50° C. then 8-(3-chloropropyl)-2-(trifluoromethyl)-7,8,9,10-tetrahydro-6H-[1,3]oxazolo[4,5-g][3]benzazepine (40 mg) was added and the resulting mixture was shaken at 50° C. for over night. After cooling the resin was filtered off, washed with methanol (2 ml) and then the solvent was removed under reduced pressure. Purifications were carried out using preparative chromatography in the following condition:

### Column: X Terra MS C18 5 mm, 100×19 mm

Mobile phase: A: NH4HCO3 sol. 10 mM, pH10; B: CH3CN
Gradient: 30% (B) for 1 min, from 30% (B) to 95% (B) in 9 min, 95% (B) for 3 min
Flow rate: 17 ml/min
UV wavelength range: 210-350 nm
Mass range: 100-900 amu, ionization: ES+.

Then solvent was removed under reduced pressure to give title compounds as formate salts. The residues were taken up with methanol (1 ml) and loaded on SCX SPE cartridges (1 g), washed with methanol (3 ml) and eluted with a 2N ammonia solution in methanol (3 ml) then solvent was removed under reduced pressure to give title compound (40 mg) as free base.

### HPLC: Analytical

### Column: X Terra MS C18 5 mm, 50×4.6 mm

Mobile phase: A: NH4HCO3 sol. 10 mM, pH10; B: CH3CN
Gradient: 0% (B) for 1 min, from 30% (B) to 95% (B) in 9 min, 95% (B) for 3 min
Flow rate: 1 ml/min
UV wavelength range: 210-350 nm
Mass range: 100-900 amu, ionization: ES+

MS (m/z): 478 [MH]⁺; retention time: 6.89 min.

### EXAMPLE 16

### 8-(3-{([4-methyl-5-(6-methyl-3-pyridinyl)-4H-1,2,4-triazol-3-yl]thio}propyl)-2-(trifluoromethyl)-7,8,9,10-tetrahydro-6H-[1,3]oxazolo[4,5-g][3]benzazepine

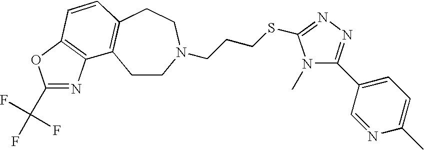

The title compound was prepared in analogy to Example 15 from 8-(3-chloropropyl)-2-(trifluoromethyl)-7,8,9,10-tetrahydro-6H-[1,3]oxazolo[4,5-g][3]benzazepine (40 mg) and 4-methyl-5-(6-methyl-3-pyridinyl)-2,4-dihydro-3H-1,2,4-triazole-3-thione (25 mg) to give the title compound (44 mg) as free base.

MS (m/z): 477 [MH]⁺. Retention time: 6.04 min

### EXAMPLE 17

### 8-(3-{[4-methyl-5-(2-methyl-3-pyridinyl)-4H-1,2,4-triazol-3-yl]thio}propyl)-2-(trifluoromethyl)-7,8,9,10-tetrahydro-6H-[1,3]oxazolo[4,5-g][3]benzazepine

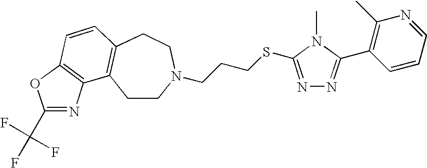

The title compound was prepared in analogy to Example 15 from 8-(3-chloropropyl)-2-(trifluoromethyl)-7,8,9,10-tetrahydro-6H-[1,3]oxazolo[4,5-g][3]benzazepine (40 mg) and 4-methyl-5-(2-methyl-3-pyridinyl)-2,4-dihydro-3H-1,2,4-triazole-3-thione (25 mg) to give the title compound (40 mg) as free base.

MS (m/z): 477 [MH]⁺. Retention time: 5.80 min

### EXAMPLE 18

### 8-{3-[(4-methyl-5-phenyl-4H-1,2,4-triazol-3-yl)thio]propyl}-2-(trifluoromethyl)-7,8,9,10-tetrahydro-6H-[1,3]oxazolo[4,5-g][3]benzazepine

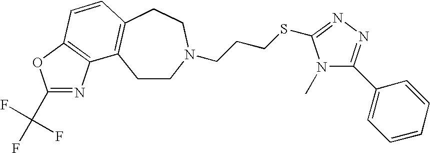

The title compound was prepared in analogy to Example 15 from 8-(3-chloropropyl)-2-(trifluoromethyl)-7,8,9,10-tetrahydro-6H-[1,3]oxazolo[4,5-g][3]benzazepine (40 mg) and 4-methyl-5-phenyl-2,4-dihydro-3H-1,2,4-triazole-3-thione (23 mg) to give the title compound (42 mg) as free base.

NMR (¹H, CD₃OD): δ 7.7 (m, 2H), 7.59 (m, 3H), 7.49 (d, 1H), 7.38 (d, 1H), 3.7 (s, 3H), 3.38 (m, 2H), 3.29 (m, 2H), 3.11 (m, 2H), 2.72 (m, 6H)), 2.02 (m, 2H). MS (m/z): 462 [MH]⁺. Retention time: 7.20 min

### EXAMPLE 19

### 8-(3-{[5-(2,4-dimethyl-1,3-thiazol-5-yl)-4-methyl-4H-1,2,4-triazol-3-yl]thio}propyl)-2-(trifluoromethyl)-7,8,9,10-tetrahydro-6H-[1,3]oxazolo[4,5-g][3]benzazepine

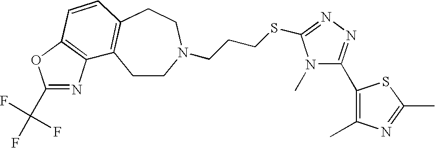

The title compound was prepared in analogy to Example 15 from 8-(3-chloropropyl)-2-(trifluoromethyl)-7,8,9,10-tetrahydro-6H-[1,3]oxazolo[4,5-g][3]benzazepine (40 mg) and 5-(2,4-dimethyl-1,3-thiazol-5-yl)-4-methyl-2,4-dihydro-3H-1,2,4-triazole-3-thione (27 mg) to give the title compound (44 mg) as free base.

MS (m/z): 497 [MH]⁺. Retention time: 6.35 mins.

### EXAMPLE 20

### 2-Methyl-8-(3-{[4-methyl-5-(2-methyl-5-quinolinyl)-4H-1,2,4-triazol-3-yl]thio}propyl)-7,8,9,10-tetrahydro-6H-[1,3]thiazolo[5,4-g][3]benzazepine hydrochloride

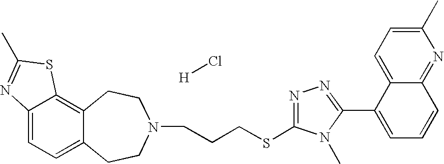

The title compound was prepared in analogy to General Procedure 1 from 2-methyl-7,8,9,10-tetrahydro-6H-[1,3]thiazolo[5,4-g][3]benzazepine (0.13 mmol) and 5-{5-[(3-chloropropyl)thio]-4-methyl-4H-1,2,4-triazol-3-yl}-2-methylquinoline to give the title compound (0.027 mmol) as a colourless slightly hygroscopic solid.

NMR (¹H, DMSO): δ 10.55 (vbs, 1H), 8.14 (2d, 2H), 7.88 (d, 1H), 7.7 (m, 2H), 7.49-7.36 (2d, 2H), 3.8-3.0 (vbm, 12H), 3.43 (s, 3H), 2.78-2.69 (2s, 6H), 2.27 (quint, 2H).

MS (m/z): 515 [MH]⁺.

### EXAMPLE 21

### 2-ethyl-8-(3-{[4-methyl-5-(2-methyl-5-quinolinyl)-4H-1,2,4-triazol-3-yl]thio}propyl)-7,8,9,10-tetrahydro-6H-[1,3]thiazolo[5,4-g][3]benzazepine hydrochloride

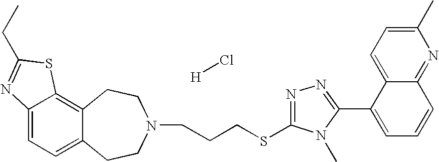

The title compound was prepared in analogy to General Procedure 1 from: 2-ethyl-7,8,9,10-tetrahydro-6H-[1,3]thiazolo[5,4-g][3]benzazepine (0.43 mmol) and 5-{5-[(3-chloropropyl)thio]-4-methyl-4H-1,2,4-triazol-3-yl}-2-methylquinoline (0.52 mmol) to give the title compound (37 mg) as a colourless slightly hygroscopic solid.

NMR (¹H, CD₃OD): δ 8.26 (m, 2H), 7.99 (t, 1H), 7.83-7.80 (2d, 2H), 7.82 (s, 1H), 7.6 (d, 1H), 7.43 (d, 1H), 4-3.1 (vbm, 12H), 3.55 (s, 3H), 3.18 (quart, 2H), 2.82 (s, 3H), 2.43 (quint, 2H), 1.48 (t, 3H). MS (m/z): 529 [MH]⁺.

### EXAMPLE 22

### 2-ethyl-8-(3-{[4-methyl-5-(4-methyl-1,3-oxazol-5-yl)-4H-1,2,4-triazol-3-yl]thio}propyl)-7,8,9,10-tetrahydro-6H-[1,3]thiazolo[5,4-g][3]benzazepine hydrochloride

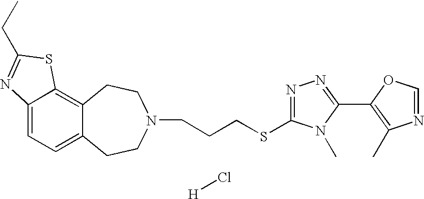

The title compound was prepared in analogy to General Procedure 1 from 2-ethyl-7,8,9,10-tetrahydro-6H-[1,3]thiazolo[5,4-g][3]benzazepine (0.14 mmol) and 3-[(3-Chloropropyl)thio]-4-methyl-5-(4-methyl-1,3-oxazol-5-yl)-4H-1,2,4-triazole (0.16 mmol) to give the title compound (7 mg) as a colourless slightly hygroscopic solid.

NMR (¹H, CD₃OD): δ 8.4 (s, 1H), 7.8 (d, 1H), 7.43 (d, 1H), 4-3.1 (vbm, 8H), 3.81 (s, 3H), 3.48-3.4 (2t, 4H), 3.19 (quart, 2H), 2.47 (s, 3H), 2.35 (quint, 2H), 1.49 (t, 3H).

MS (m/z): 469 [MH]⁺.

1. A compound of formula (I) or a salt thereof: 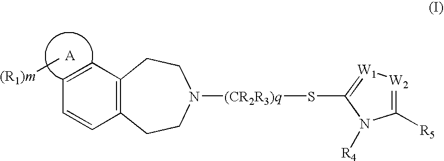
wherein
A is a 5 or 6 membered heteroaromatic ring or a 5 or 6 membered heterocyclic ring;
m is 0, 1, 2 or 3;
R₁ is independently halogen, oxo, hydroxy, cyano, nitro, C₁₋₄alkyl, haloC₁₋₄alkyl, C₃₋₆cycloalkyl, C₁₋₄alkoxy, haloC₁₋₄alkoxy, C₁₋₄alkoxyC₁₋₄alkoxy, C₁₋₄alkylenedioxy, C₁₋₄alkylthio, C₁₋₄alkoxyC₁₋₄alkyl, C₃₋₆cycloalkylC₁₋₄alkoxy, C₃₋₆cycloalkylC₁₋₄alkyl, C₁₋₄alkanoyl, C₁₋₄alkoxycarbonyl, C₁₋₄alkoxycarbonylC₁₋₄alkyl, C₁₋₄alkylsulfonyl, C₁₋₄alkylsulfonyloxy, haloC₁₋₄alkylsulfonyl, haloC₁₋₁₄alkylsulfonyloxy, C₁₋₁₄alkylsulfonylC₁₋₄alkyl, C₁₋₄alkylsulfonamido, C₁₋₄alkylsulfonamidoC₁₋₄alkyl, heterocyclyl, aryl, arylC₁₋₄alkoxy, aryloxy, arylthio, arylmethyl, aroyl, aryloxymethyl, arylsulfonyl, aryl-NR′— (wherein R′ is hydrogen or C₁₋₄alkyl), arylsulfonyloxy, arylsulfonylC₁₋₄alkyl, arylsulfonamido, arylcarboxamido, arylsulfonamidoC₁₋₄alkyl, arylcarboxamidoC₁₋₄alkyl, aroylC₁₋₄alkyl, arylC₁₋₄alkanoyl, a group NR₆R₇, R₆CON(R₇)(CH₂)ᵣ, R₆R₇NCO(CH₂)ᵣ or R₆R₇NSO₂(CH₂)ᵣ (in which r is 0, 1, 2, 3 or 4, and each of R₆ and R₇ is independently hydrogen or C₁₋₄alkyl, or in the groups NR₆R₇, R₆CON(R₇)(CH₂)ᵣ, R₆R₇NCO(CH₂)ᵣ and R₆R₇NSO₂(CH₂)ᵣ, R₆CONR₇ or NR₆R₇ together form a 4-, 5-, 6- or 7-membered azacyclic group optionally containing one additional O, N or S atom in the azacycle and having 3-8 carbon atoms (including the carbon atoms contained in any optional substituent(s) of the azacycle)); wherein in any group containing an aryl moiety, the aryl moiety is optionally substituted by one, two or three substituents selected from the group consisting of halogen, hydroxy, cyano, nitro, amino, C₁₋₄alkyl, haloC₁₋₄alkyl, C₁₋₄alkoxy, haloC₁₋₄alkoxy, C₁₋₄alkylenedioxy, C₁₋₄alkanoyl, C₁₋₄alkylsulfonyl, haloC₁₋₄alkylsulfonyl, C₁₋₄alkylamino, C₁₋₄dialkylamino, R₈R₉NCO (in which R₈ and R₉ are independently hydrogen or C₁₋₄alkyl, or R₈R₉N together form a 4-, 5-, 6- or 7-membered azacyclic group optionally containing one additional O, N or S atom in the azacycle and having 3-8 carbon atoms (including the carbon atoms contained in any optional substituent(s) of the azacycle));
R₂ and R₃ are independently hydrogen or methyl;
q is 2, 3 or 4;
W₁ and W₂ are independently N, CH or —C(C₁₋₄alkyl)-;
R₄ is hydrogen or C₁₋₄alkyl;
R₅ is a group of the formula (a) or (b):
-z (a)
—(CR₁₀R₁₁)ₜZ (b)
wherein
Z is C₁₋₄alkyl, haloC₁₋₄alkyl, C₃₋₆cycloalkyl, phenyl, heterocyclyl, a 5- or 6-membered heteroaromatic group or a 8- to 11-membered bicyclic group, any of which is optionally substituted by 1, 2, 3 or 4 substituents selected from the group consisting of: halogen, hydroxy, oxo, cyano, nitro, C₁₋₄alkyl, C₁₋₄alkoxy, haloC₁₋₄alkyl, haloC₁₋₄alkoxy, C₁₋₄alkylenedioxy, C₁₋₄alkanoyl, C₁₋₄alkylsulfonyl, C₁₋₄alkylsulfonyloxy, haloC₁₋₄alkylsulfonyl, haloC₁₋₄alkylsulfonyloxy, C₁₋₄alkylsulfinyl, C₁₋₄alkylthio, R₁₂SO₂NR₁₃—, R₁₂R₁₃NSO₂—, R₁₂R₁₃N—, R₁₂R₁₃NCO—, R₁₂CONR₁₃— and a 5- or 6-membered heteroaromatic group which is optionally substituted by one or two groups selected from C₁₋₂alkyl, haloC₁₋₂alkyl and R₁₂R₁₃N—; and wherein substituents positioned ortho to one another may be linked to form a 5- or 6-membered ring;
R₁₀ and R₁₁ are independently hydrogen or C₁₋₄alkyl and t is 1, 2, 3 or 4, or —(CR₁₀R₁₁)ₜ— forms a C₃₋₆cycloalkylene linker; and
R₁₂ and R₁₃ are independently hydrogen or C₁₋₄alkyl, or R₁₂ and R₁₃ together form C₃₋₆alkylene.

2. A compound as claimed in claim 1, wherein m is 0 or 1.

3. A compound as claimed in claim 1, wherein R₁ is halogen, oxo, cyano, C₁₋₄alkyl (such as methyl, ethyl, propyl, isopropyl, butyl, tert-butyl), haloC₁₋₄alkyl (such as —CF₃, CF₃CH₂— or pentafluoroethyl), acetyl, trifluoromethoxy, C₃₋₆cycloalkylC₁₋₄alkyl (such as cyclopropylmethyl), C₃₋₆cycloalkyl (such as cyclopropyl), C₁₋₄alkylsulfonyl, C₁₋₄alkylsulfonyloxy, R₆R₇NSO₂ (where each of R₆ and R₇ is independently hydrogen or C₁₋₄alkyl or R₆R₇N together form a 4-, 5-, 6- or 7-membered azacyclic group optionally containing one additional O, N or S atom in the azacycle and having 3-8 carbon atoms), a heterocyclyl, or a 5- or 6-membered heteroaromatic group which is optionally substituted by one or two substituents selected from: halogen, cyano, C₁₋₂alkyl (e.g. methyl), haloC₁₋₂alkyl (e.g. trifluoromethyl), C₁₋₂alkoxy (e.g. methoxy), C₁₋₂alkylenedioxy (e.g. methylenedioxy), C₁₋₃alkanoyl (e.g. acetyl), C₂alkanoylamino (e.g. acetylamino), haloC₁alkylsulfonyl (e.g. trifluoromethylsulfonyl) and methylsulfonyl.

4. A compound as claimed in claim 1, wherein R₂ and R₃ are hydrogen at each occurrence.

5. A compound as claimed in claim 1, wherein q is 2 or 3.

6. A compound as claimed in claim 1, wherein W₁ and W₂ are both N.

7. A compound as claimed in claim 1, wherein R₄ is hydrogen or methyl.

8. A compound as claimed in claim 1, wherein R₅ is a group of formula (a) as defined in claim 1.

9. A compound as claimed in claim 1, which is:
2-methyl-8-(3-{[4-methyl-5-(2-methyl-5-quinolinyl)-4H-1,2,4-triazol-3-yl]thio}propyl)-7,8,9,10-tetrahydro-6H-[1,3]oxazolo[4,5-g][3]benzazepine
2-ethyl-8-(3-{[4-methyl-5-(2-methyl-5-quinolinyl)-4H-1,2,4-triazol-3-yl]thio}propyl)-7,8,9,10-tetrahydro-6H-[1,3]oxazolo[4,5-g][3]benzazepine
2-ethyl-8-(3-{[4-methyl-5-(4-methyl-1,3-oxazol-5-yl)-4H-1,2,4-triazol-3-yl]thio}propyl)-7,8,9,10-tetrahydro-6H-[1,3]oxazolo[4,5-g][3]benzazepine
8-(3-{[4-methyl-5-(2-methyl-5-quinolinyl)-4H-1,2,4-triazol-3-yl]thio}propyl)-2-(trifluoromethyl)-7,8,9,10-tetrahydro-6H-[1,3]oxazolo[4,5-g][3]benzazepine
8-(3-{[4-methyl-5-(4-methyl-1,3-oxazol-5-yl)-4H-1,2,4-triazol-3-yl]thio}propyl)-2-(trifluoromethyl)-7,8,9,10-tetrahydro-6H-[1,3]oxazolo[4,5-g][3]benzazepine
2-ethyl-8-(3-{[4-methyl-5-(2-methyl-5-quinolinyl)-4H-1,2,4-triazol-3-yl]thio}propyl)-7,8,9,10-tetrahydro-6H-[1,3]thiazolo[5,4-g][3]benzazepine
2-(1,3-dimethyl-1H-pyrazol-5-yl)-8-(3-{[4-methyl-5-(2-methyl-5-quinolinyl)-4H-1,2,4-triazol-3-yl]thio}propyl)-7,8,9,10-tetrahydro-6H-[1,3]oxazolo[4,5-g][3]benzazepine
2-(1,3-dimethyl-1H-pyrazol-5-yl)-8-(3-{[4-methyl-5-(4-methyl-1,3-oxazol-5-yl)-4H-1,2,4-triazol-3-yl]thio}propyl)-7,8,9,10-tetrahydro-6H-[1,3]oxazolo[4,5-g][3]benzazepine
8-(3-{[4-methyl-5-(2-methyl-5-quinolinyl)-4H-1,2,4-triazol-3-yl]thio}propyl)-2-(pentafluoroethyl)-7,8,9,10-tetrahydro-6H-[1,3]oxazolo[4,5-g][3]benzazepine
8-(3-{[4-methyl-5-(4-methyl-1,3-oxazol-5-yl)-4H-1,2,4-triazol-3-yl]thio}propyl)-2-(pentafluoroethyl)-7,8,9,10-tetrahydro-6H-[1,3]oxazolo[4,5-g][3]benzazepine
8-(3-{[4-methyl-5-(2-methyl-5-quinolinyl)-4H-1,2,4-triazol-3-yl]thio}propyl)-2-(trifluoromethyl)-7,8,9,10-tetrahydro-6H-[1,3]oxazolo[5,4-g][3]benzazepine
8-(3-{[4-methyl-5-(4-methyl-1,3-oxazol-5-yl)-4H-1,2,4-triazol-3-yl]thio}propyl)-2-(trifluoromethyl)-7,8,9,10-tetrahydro-6H-[1,3]oxazolo[5,4-g][3]benzazepine
2-(1,1-difluoroethyl)-8-(3-{[4-methyl-5-(4-methyl-1,3-oxazol-5-yl)-4H-1,2,4-triazol-3-yl]thio}propyl)-7,8,9,10-tetrahydro-6H-[1,3]oxazolo[4,5-g][3]benzazepine
2-(1,1-difluoroethyl)-8-(3-{[4-methyl-5-(2-methyl-5-quinolinyl)-4H-1,2,4-triazol-3-yl]thio}propyl)-7,8,9,10-tetrahydro-6H-[1,3]oxazolo[5,4-g][3]benzazepine
2-(1,1-difluoroethyl)-8-(3-{[4-methyl-5-(4-methyl-1,3-oxazol-5-yl)-4H-1,2,4-triazol-3-yl]thio}propyl)-7,8,9,10-tetrahydro-6H-[1,3]oxazolo[5,4-g][3]benzazepine
8-(3-{[4-methyl-5-(5-methyl-2-pyrazinyl)-4H-1,2,4-triazol-3-yl]thio}propyl)-2-(trifluoromethyl)-7,8,9,10-tetrahydro-6H-[1,3]oxazolo[4,5-g][3]benzazepine
8-(3-{[4-methyl-5-(6-methyl-3-pyridinyl)-4H-1,2,4-triazol-3-yl]thio}propyl)-2-(trifluoromethyl)-7,8,9,10-tetrahydro-6H-[1,3]oxazolo[4,5-g][3]benzazepine
8-(3-{[4-methyl-5-(2-methyl-3-pyridinyl)-4H-1,2,4-triazol-3-yl]thio}propyl)-2-(trifluoromethyl)-7,8,9,10-tetrahydro-6H-[1,3]oxazolo[4,5-g][3]benzazepine
8-{3-[(4-methyl-5-phenyl-4H-1,2,4-triazol-3-yl)thio]propyl}-2-(trifluoromethyl)-7,8,9,10-tetrahydro-6H-[1,3]oxazolo[4,5-g][3]benzazepine
8-(3-{[5-(2,4-dimethyl-1,3-thiazol-5-yl)-4-methyl-4H-1,2,4-triazol-3-yl]thio}propyl)-2-(trifluoromethyl)-7,8,9,10-tetrahydro-6H-[1,3]oxazolo[4,5-g][3]benzazepine
2-methyl-8-(3-{[4-methyl-5-(2-methyl-5-quinolinyl)-4H-1,2,4-triazol-3-yl]thio}propyl)-7,8,9,10-tetrahydro-6H-[1,3]thiazolo[5,4-g][3]benzazepine
2-ethyl-8-(3-{[4-methyl-5-(4-methyl-1,3-oxazol-5-yl)-4H-1,2,4-triazol-3-yl]thio}propyl)-7,8,9,10-tetrahydro-6H-[1,3]thiazolo[5,4-g][3]benzazepine
or a salt thereof.

10. A process for preparing a compound as defined in claim 1, which process comprises:
(a) reacting a compound of formula (II): 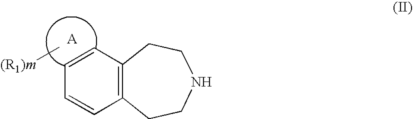
wherein R₁, m and A are as defined for formula (I), with a compound of formula (III): 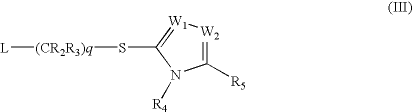
wherein R₂, R₃, q, W₁, W₂, R₄ and R₅ are as defined for formula (I), and L is a leaving group;
or
(b) reacting a compound of formula (IV): 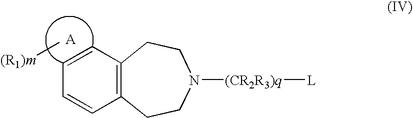
wherein A, R₁, R₂, R₃, m and q are as defined for formula (I) and L is a leaving group, with a compound of formula (V): 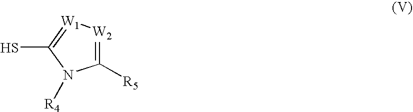
wherein W₁, W₂, R₄ and R₅ are as defined for formula (I);
and optionally thereafter for step (a) or step (b):
removing any protecting group(s); and/or
forming a salt; and/or
converting one compound of formula (I) to a different compound of formula (I).

11. A method of treating a condition for which modulation of dopamine D₃ receptors is beneficial, which comprises administering to a mammal in need thereof an effective amount of a compound as claimed in claim 1.

12. A method as claimed in claim 11, wherein the condition is substance abuse and/or drug dependency.

13. A method as claimed in claim 12, wherein the condition is craving for abused substance and/or relapse to drug seeking and drug taking behaviour.

14-20. (canceled)

21. A pharmaceutical composition comprising a compound as claimed in claim 1 and a pharmaceutically acceptable carrier.
